(19) United States
(12) Patent Application Publication
(10) Pub. No.: US 2023/0381133 A1
(43) Pub. Date: Nov. 30, 2023

(54) TAU AND AMYLOID PROTEIN LOWERING COMPOUNDS AND METHODS OF USE

(71) Applicant: Qatar Foundation for Education, Science and Community Development, Doha (QA)

(72) Inventor: Nasser Hussein Zawia, Doha (QA)

(21) Appl. No.: 18/202,525

(22) Filed: May 26, 2023

Related U.S. Application Data
(60) Provisional application No. 63/365,349, filed on May 26, 2022.

(51) Int. Cl. A61K 31/277; A61P 25/28; A61K 31/192; A61K 31/196

(52) U.S. Cl. CPC A61K 31/277; A61P 25/28; A61K 31/192; A61K 31/196

(57) ABSTRACT

Methods and compositions for treating neurodegenerative diseases in a patient in need thereof are provided. The methods include administering to the patient a therapeutically effective amount of a Tolfenamic Acid (TA) analog or a pharmaceutically acceptable salt thereof. The TA analog may be 2-(2-cyanophenylthio) benzoic acid or a pharmaceutically acceptable salt thereof.

### CROSS REFERENCE TO RELATED APPLICATIONS

This application claims the benefit of U.S. Provisional patent application 63/365,349 filed May 26, 2022, the entire contents of which is incorporated by reference herein.

### FIELD

The present disclosure relates to compounds and methods for treating neurodegenerative diseases.

### BACKGROUND

Alzheimer's Disease (AD) and Parkinson's disease are prevalent neurodegenerative diseases, affecting more than 1 in 9 people (11.3%) over the age of 65. These diseases are characterized by the accumulation of extracellular amyloid β (Aβ) plaques (APP) and intracellular neurofibrillary tau tangles (NFTs) composed of hyperphosphorylated tau) (see e.g., FIG. 1). Additionally, tau deposition is also a feature of several diseases referred to as “tauopathies,” which feature tau deposits in the absence of amyloid.

Alzheimer's disease (AD) is a chronic, neurodegenerative disorder and is the leading cause of dementia and is the 6^{th} leading cause of death. 55 million people live with dementia worldwide. This number is expected to rise to ˜140 million by 2050.

Currently, there are no known disease-modifying therapies for treating tauopathies. The existing challenges for treating tauopathies are the inability to diagnose early and to confidently discriminate between distinct tauopathies in patients, coupled with an incomplete understanding of the cellular mechanisms involved in pathogenic tau-induced neuronal death and dysfunction. Although some chemical compositions have been utilized to lower Tau and APP, most of these compositions have failed in clinical trials.

Therefore, there is a need for new and innovative methods and compounds to address the current limitations in this field.

### SUMMARY

The present disclosure provides compositions and methods for reducing Tau and APP levels to treat neurodegenerative diseases, such as Alzheimer's and chronic traumatic encephalopathy. Specifically, the disclosed embodiments target Tau and APP levels by interrupting upstream transcriptional pathways that produce these gene products. The methods aim to disable the transcription factor SP1 to interrupt the de novo synthesis of tau and APP. Previous attempts to reduce tau and APP levels have targeted downstream effects such as the final toxic aggregated protein. Additionally, the disclosed compositions and methods can also be used to treat injuries such as traumatic brain injury. Disclosed compositions and methods can also be used to treat, for example, conditions in which overexpression of APP and abundant plaques is a feature, such as Down's Syndrome.

### BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 shows neuronal characteristics of neurodegenerative disease.

FIG. 2. Dementia demographics in Qatar. People above 65 years represented 1.5 percent (43,000) of the total population Qatar This number is expected to increase to 14.2 percent (546,000) by 2050. There are an estimated 4,400 persons with dementia in Qatar. This number is expected to rise to over 40,000 by 2050.

FIG. 3. AD biomarkers. Preclinical AD, Onset of cognitive impairment, Symptomatic AD.

FIG. 4. The pathological hallmarks of AD. Accumulated amyloid β (Aβ) plaques. Neurofibrillary tangles (tau). Cognitive impairment correlates best with the burden of tau neurofibrillary tangles.

FIG. 5. Tau pathology. Hyperphosphorylation of tau is responsible for the neurofibrillary lesions.

FIG. 6. TA and its analogs. Conformations after modifications of selected functional group were superimposed on a prototype TA conformation. The tendency of each compound to bind zinc ion in gas phase was studied. Complete geometry optimizations were carried out in presence of zinc allowing the compound to reorganize to capture the ion in the most efficient way. The overall binding affinity was then viewed as a combination of stabilization gained in binding and the loss in conformational rearrangement required for efficient capture of the ion.

FIG. 7. TA and its analogs. Cytotoxicity screen. TN3 and TN7 showed better safety profiles than TA 72 h after treatment.

FIGS. 8A-8C. TA and its analogs—Sp1 ChIP_Seq. FIG. 8A shows plots for DMSO, TA, TN3 and TN7. FIGS. 8B-8C show Sp1 ChIP_Seq data.

FIGS. 9A-9C. TA and its analogs. Transcriptomics (mRNA_Seq). Volcano plots of pairwise differential gene expressions. Volcano plots of TA (FIG. 9A), TN3 (FIG. 9B) and TN7 (FIG. 9C) with DMSO.

FIG. 10. TA and its analogs. Transcriptomics (mRNA_Seq).

FIG. 11. TA and its analogs. Study design. FIG. 12. Tolfenamic acid (TN1, kCMZ605) plasma and terminal brain exposure.

FIGS. 13A-13C. TA and its analogs. Brain penetration. TN3 and TN7 can cross the blood-brain barrier. Concentration vs time plots for TA (FIG. 13A), TN3 (FIG. 13B) and TN7 (FIG. 13C)

FIGS. 14A-14C. TA and its analogs. Transcriptomics (in vivo). TA vs WT (FIG. 14A); TN3 vs WT (FIG. 14B); TN7 vs WT (FIG. 14C).

FIG. 15. TA and its analogs. Transcriptomics (in vivo).

FIGS. 16A-16B provide gene and detail information. TN3 and Tn7 target neurodegenerative pathways in the CNS.

FIG. 17. TA and its analogs. Proteomics (in vivo).

FIGS. 18A-18D show overview of synthetic strategy, lead compounds and their safety.

FIGS. 19A-19H show mode of action of compounds acting at the gene level; at transcription start sites to interfere with SP1 binding.

FIGS. 20A-20D show compounds alter expression of genes that are primarily SP1 targets.

FIGS. 21A-21E show final outcome of interfering at the gene level on the protein products by interrogating the whole proteome. The bar graph displays the outcomes on specific proteins involved in neurodegenerative diseases such as Alzheimers.

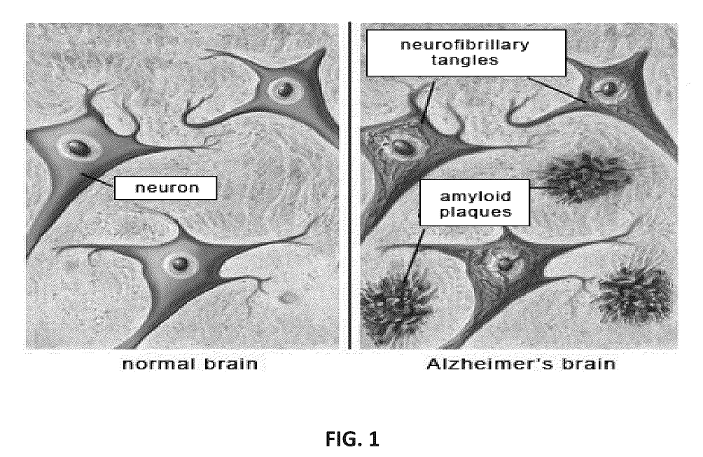

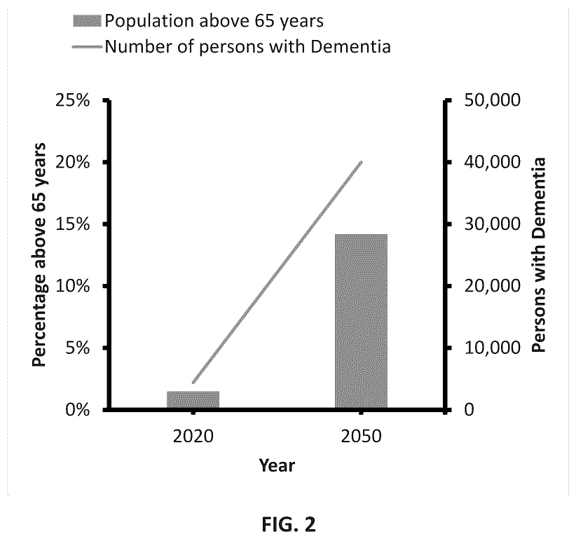

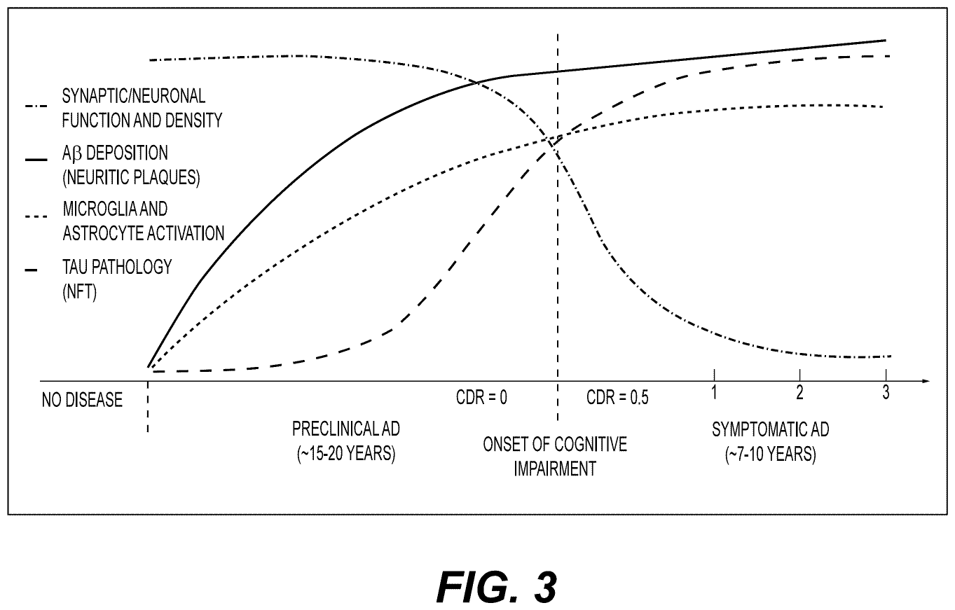

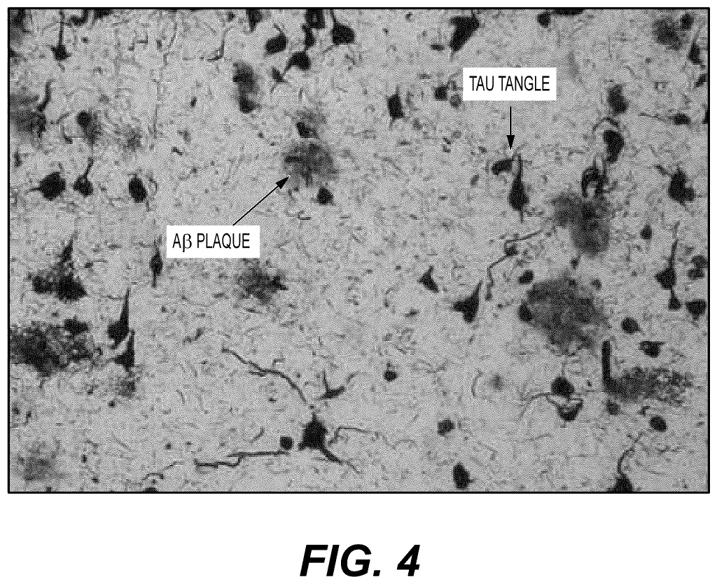

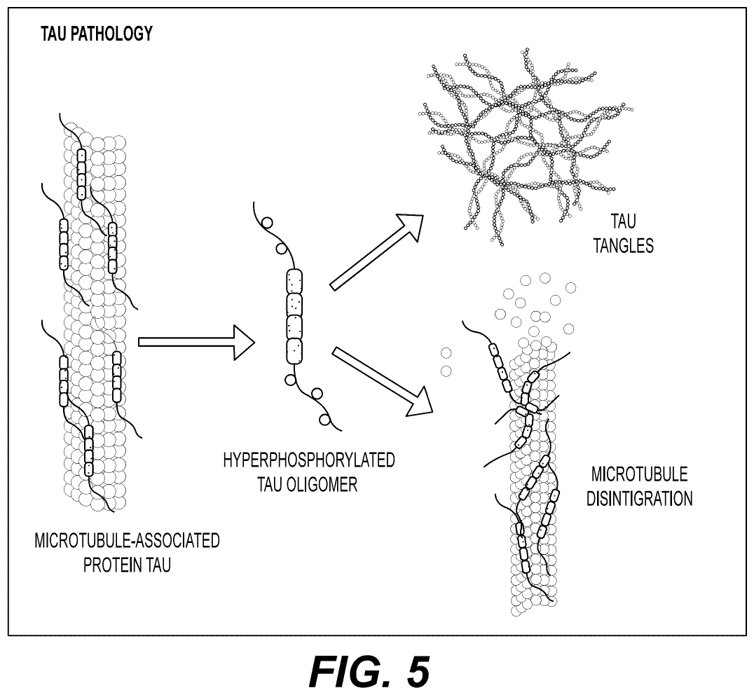

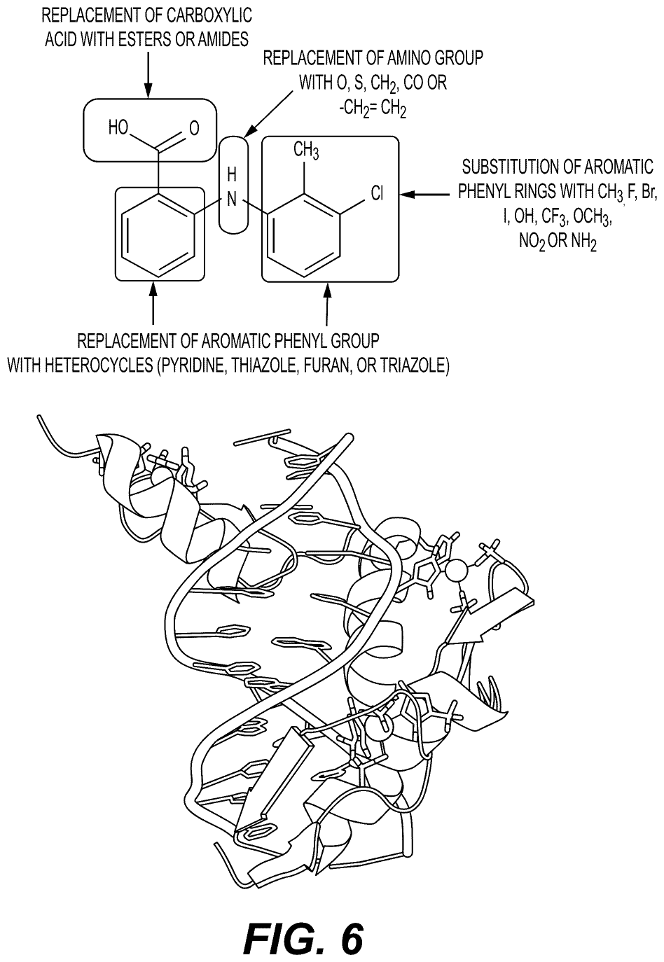

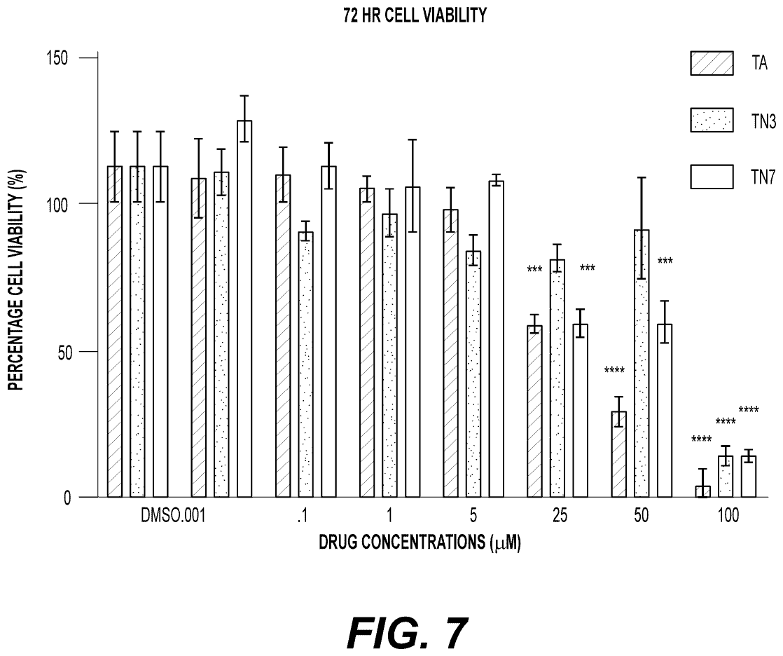

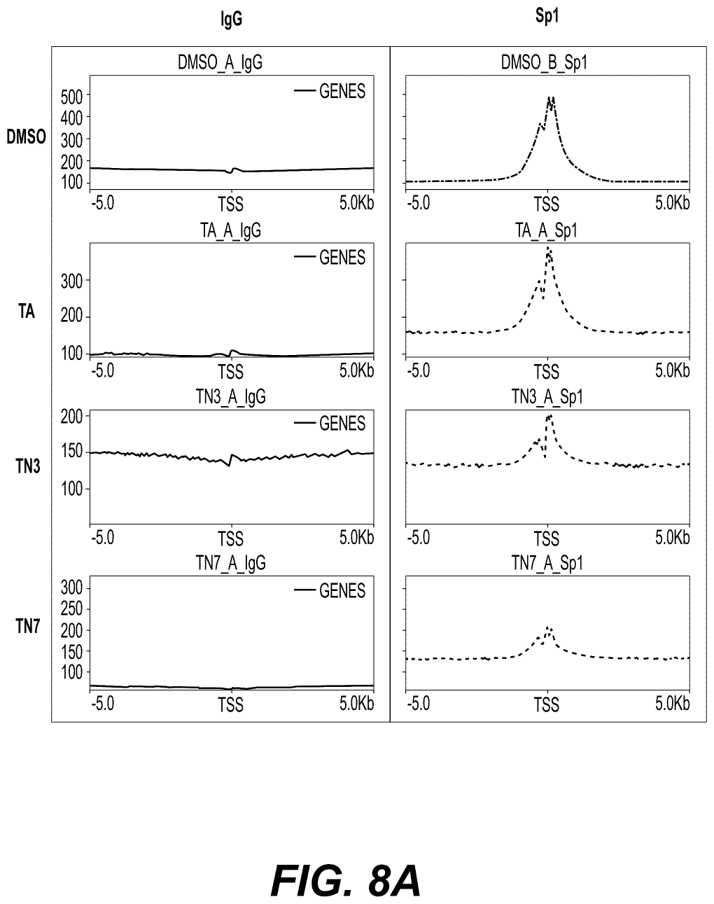

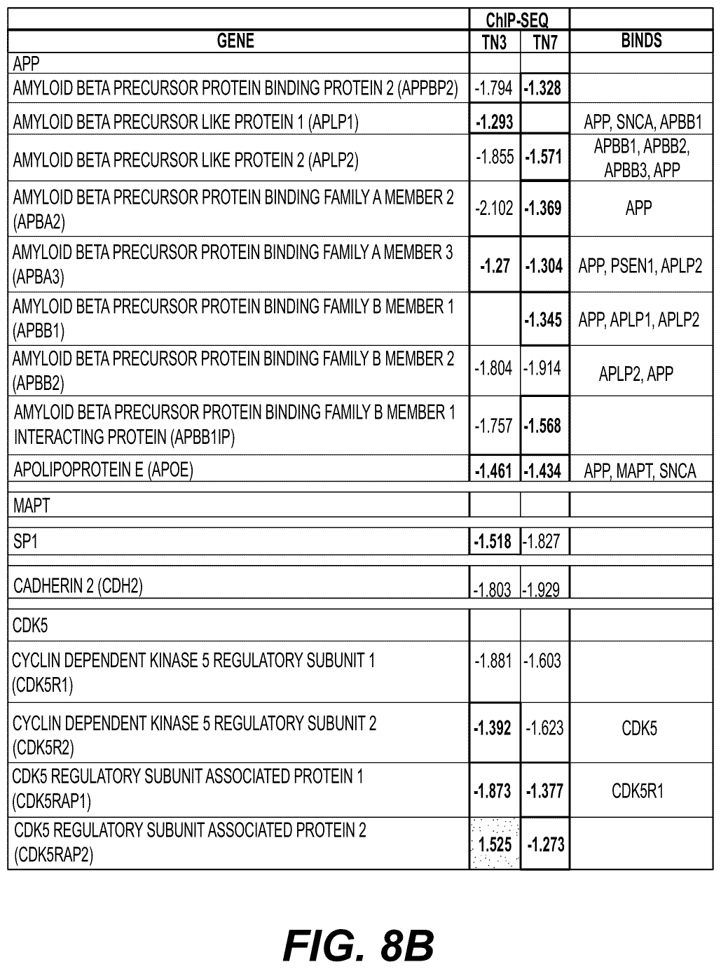

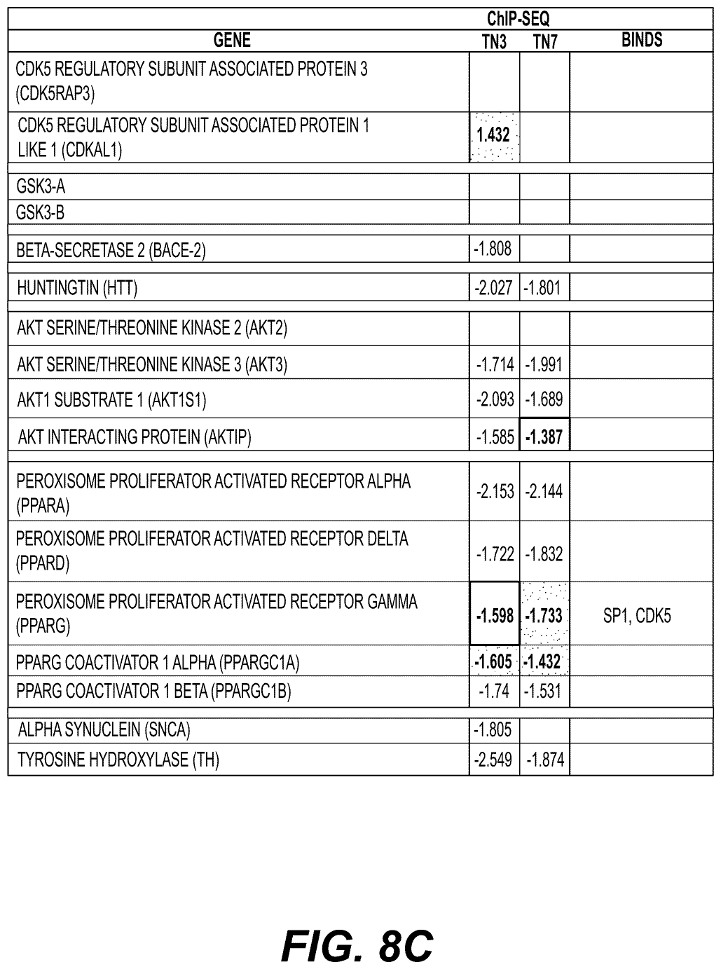

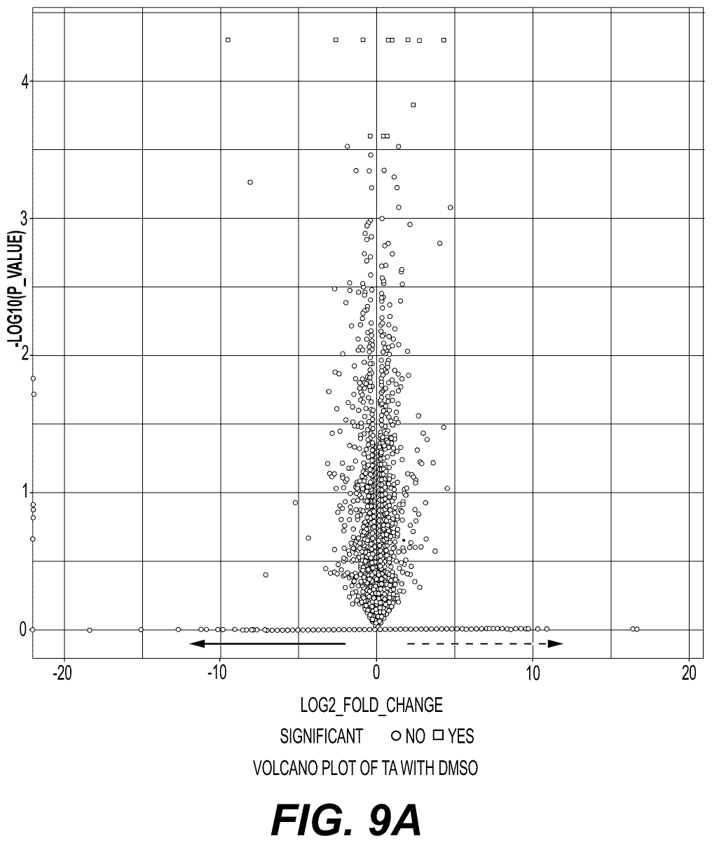

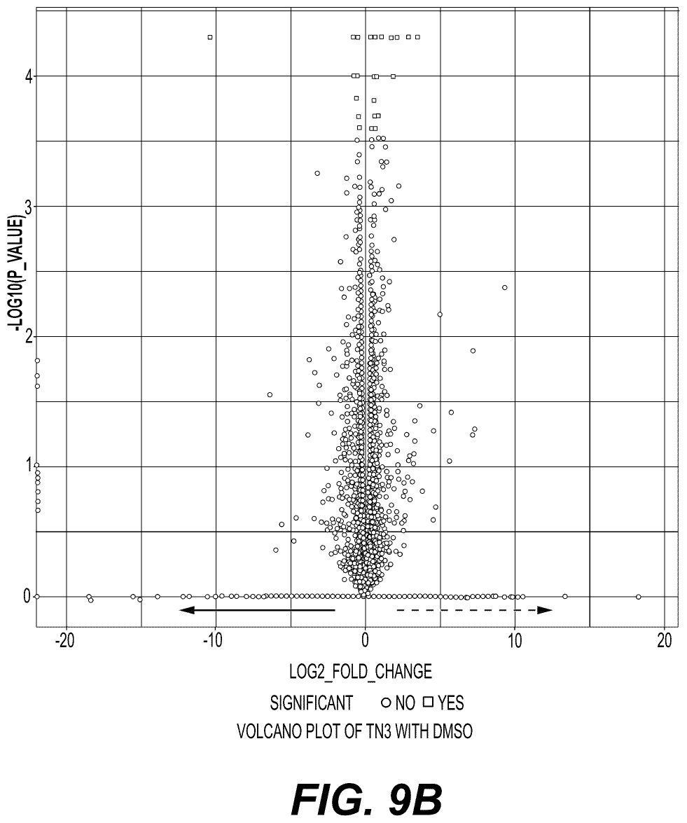

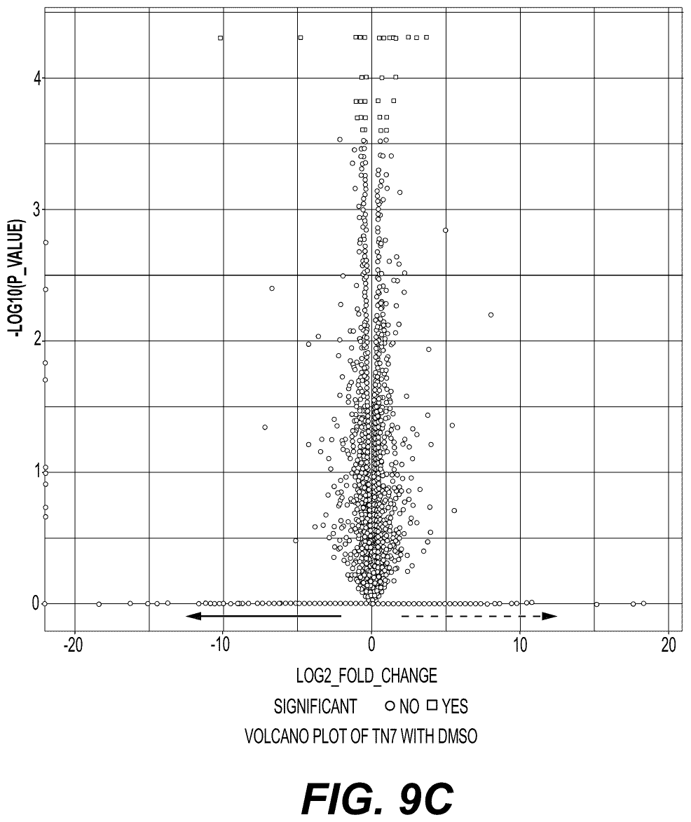

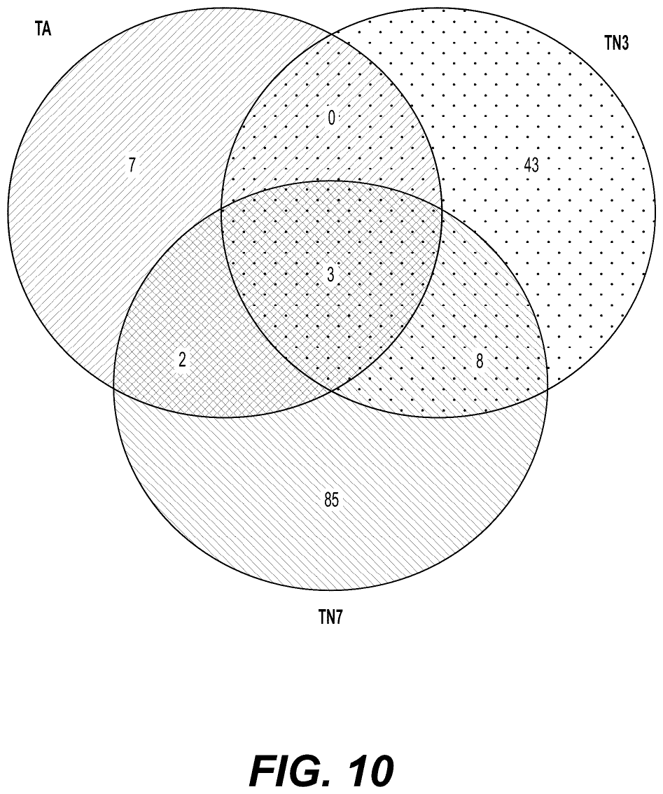

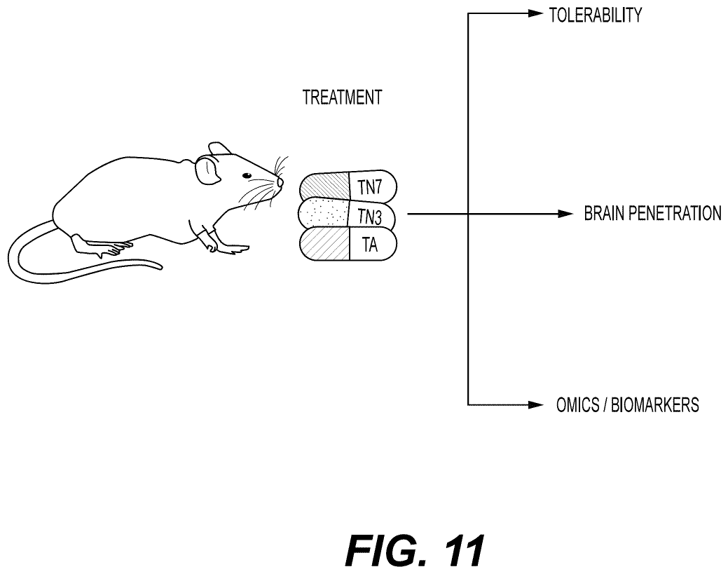

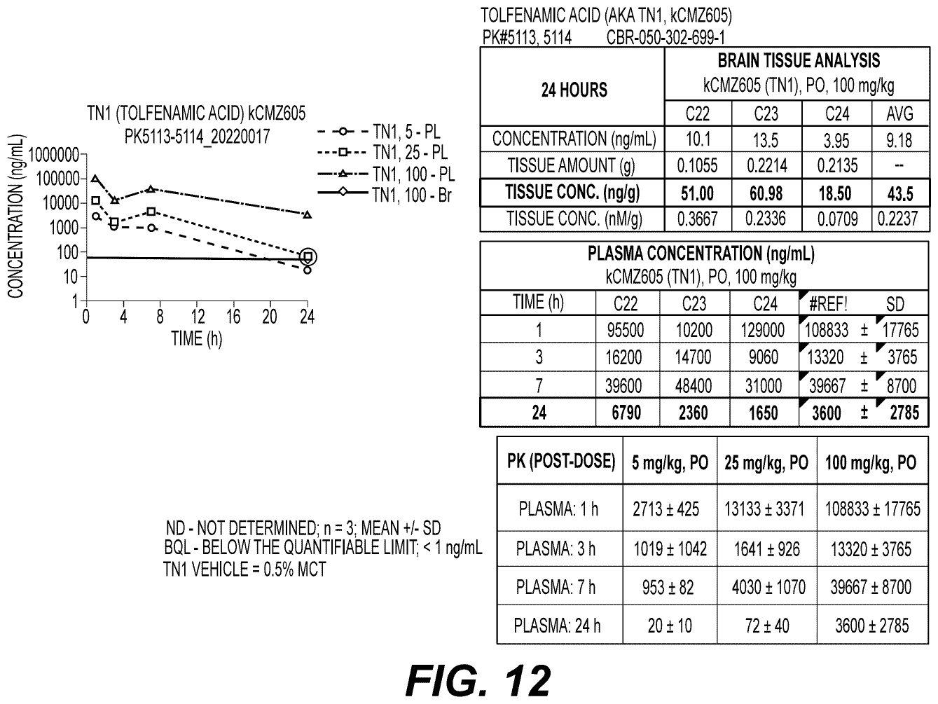

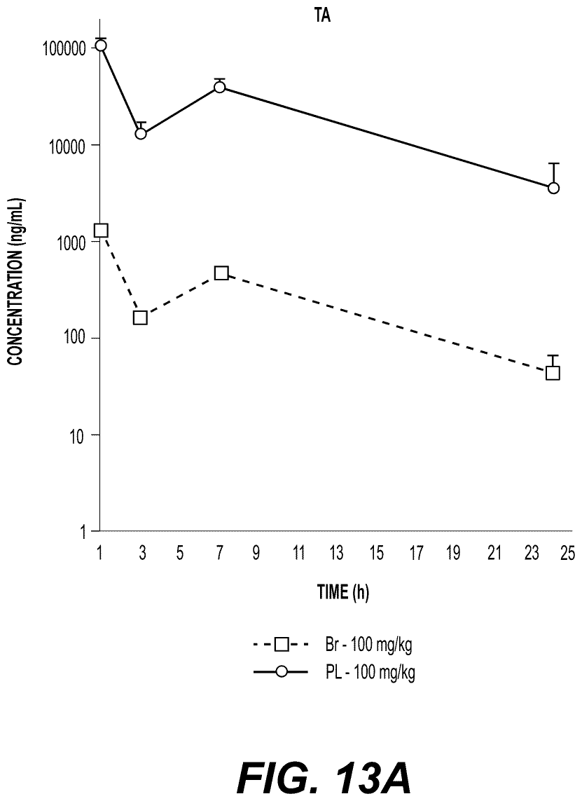

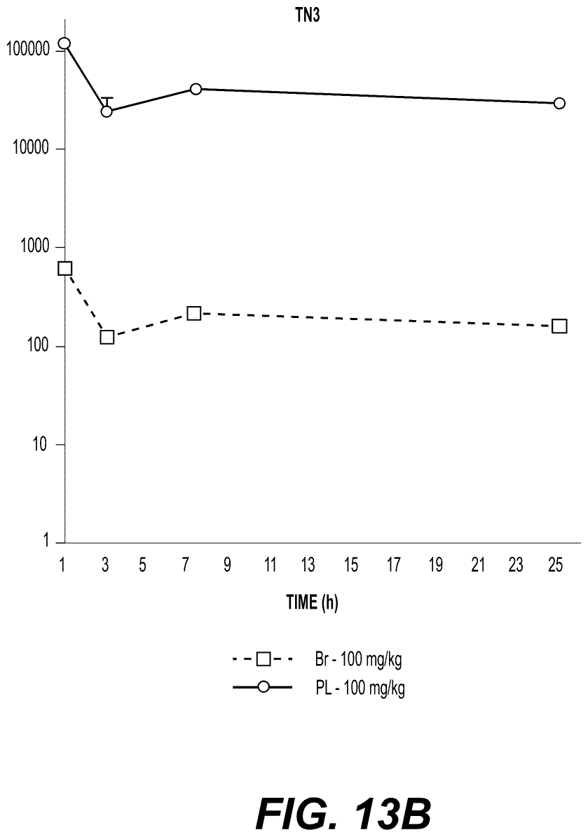

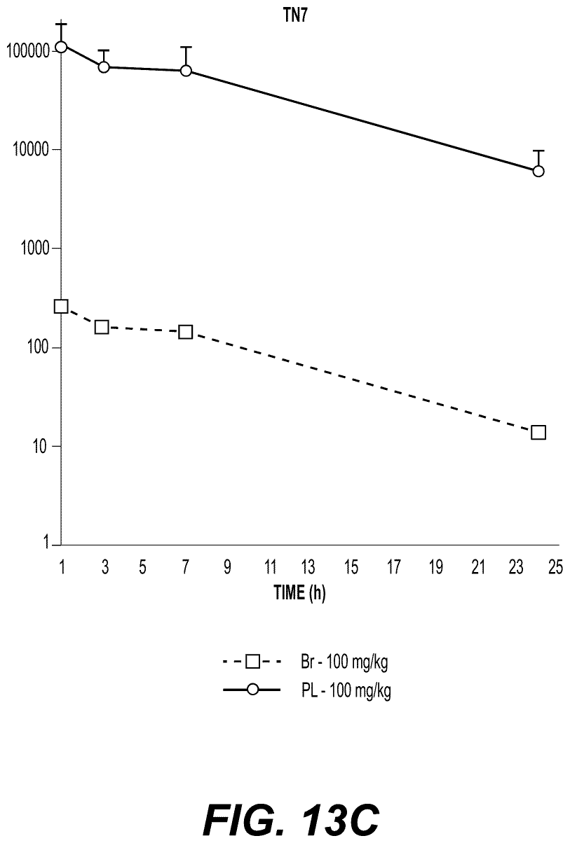

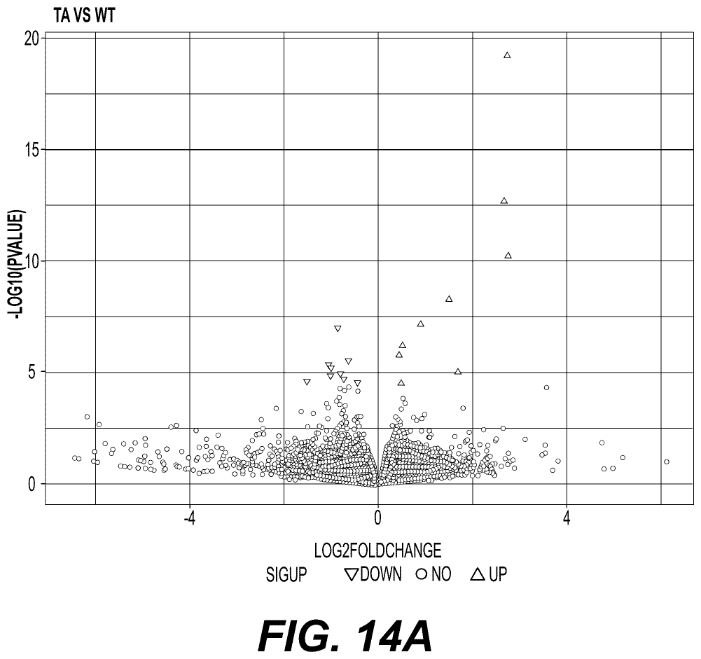

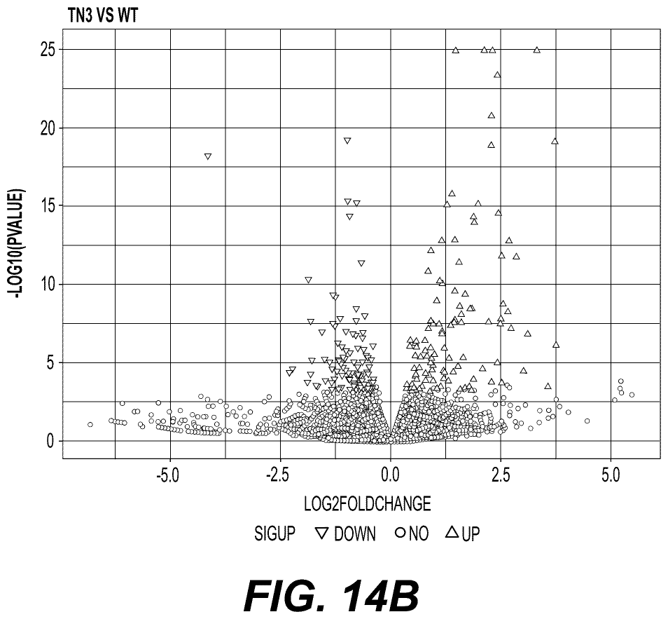

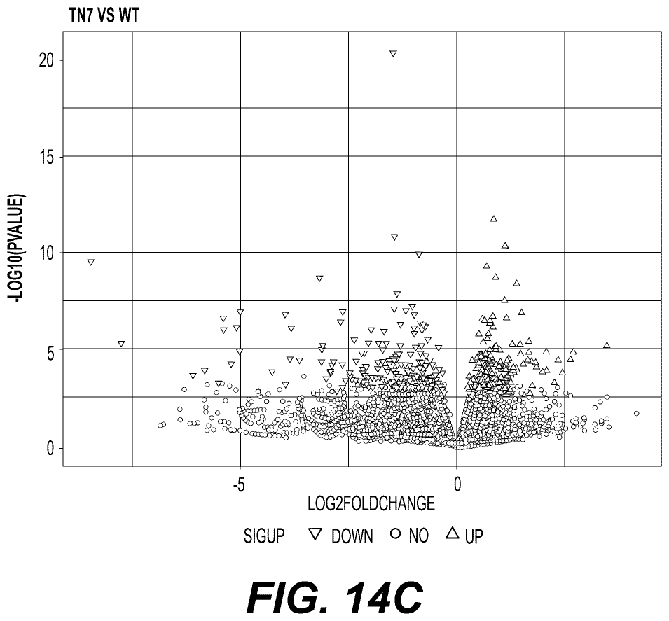

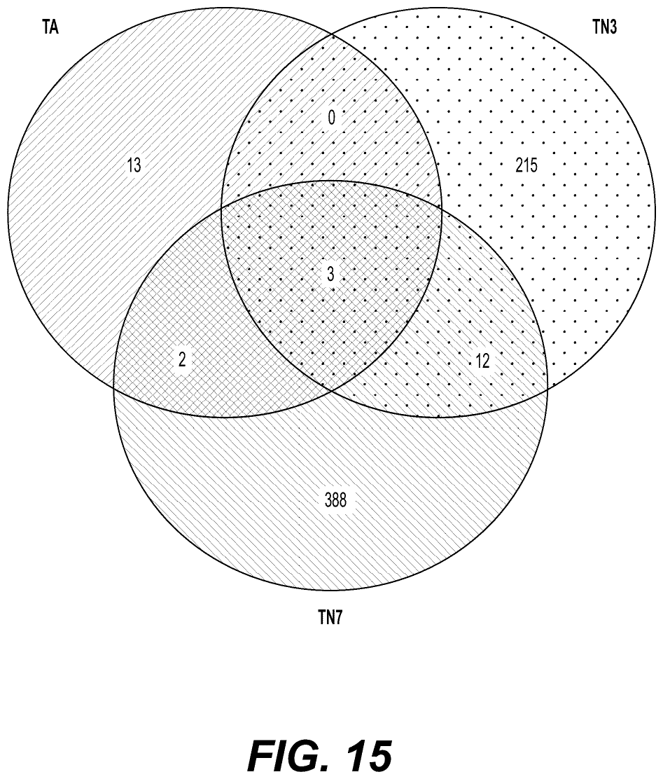

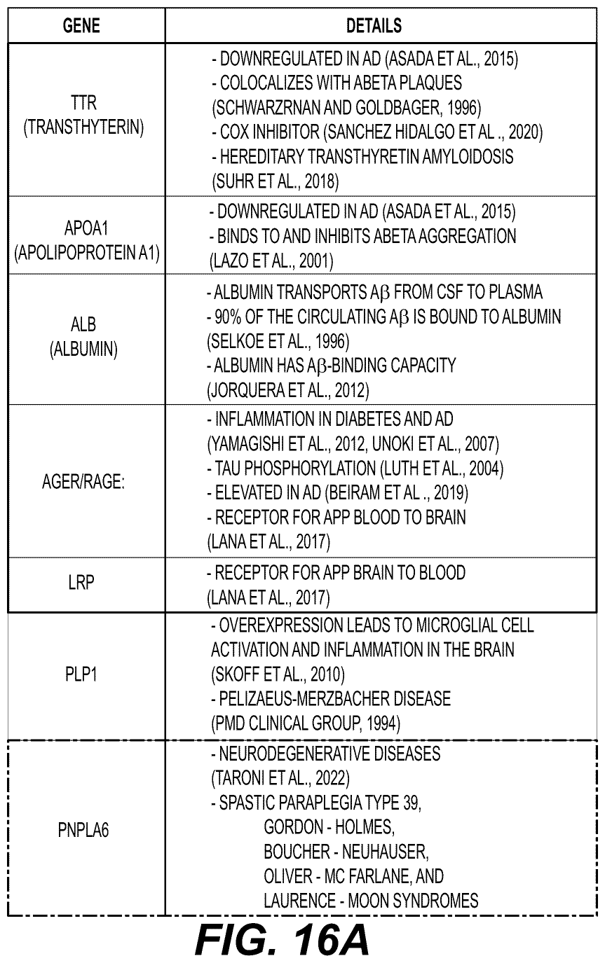

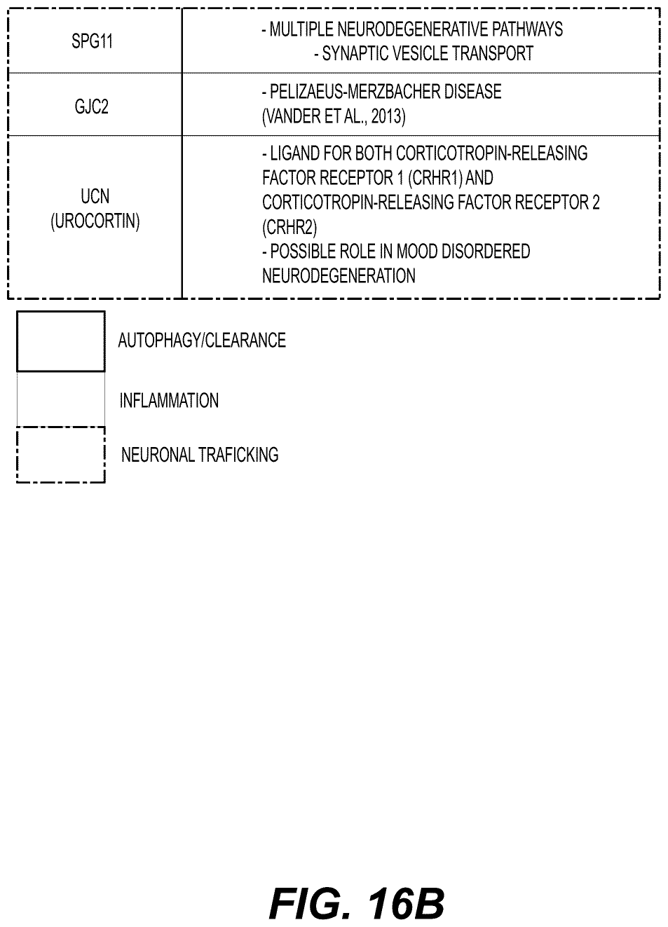

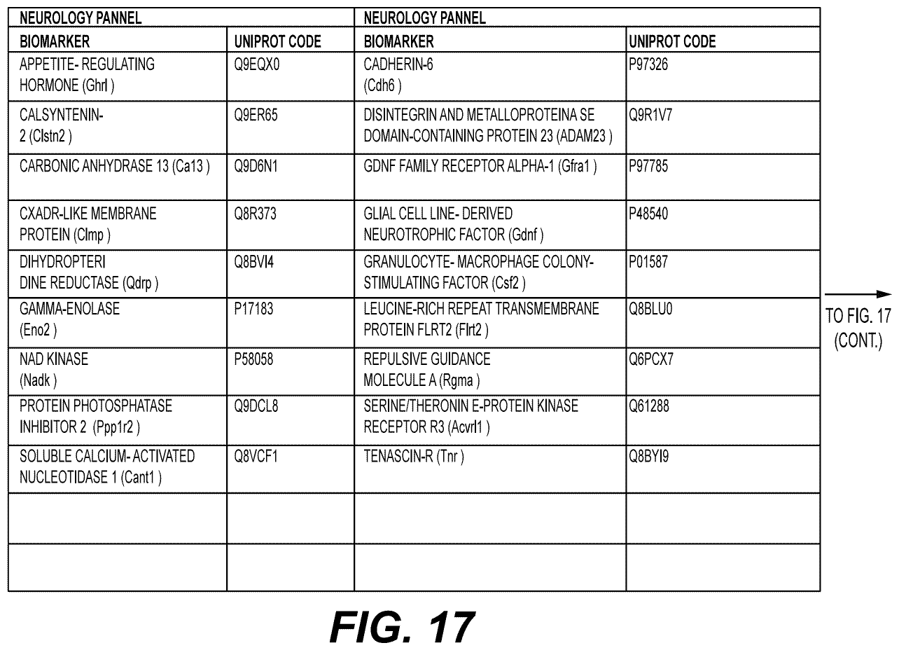

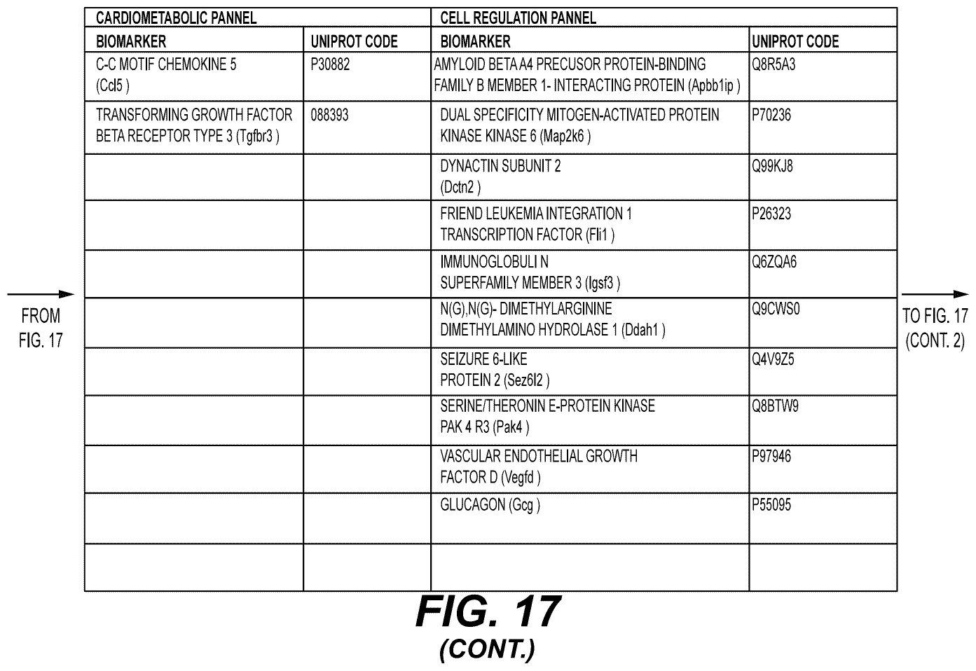

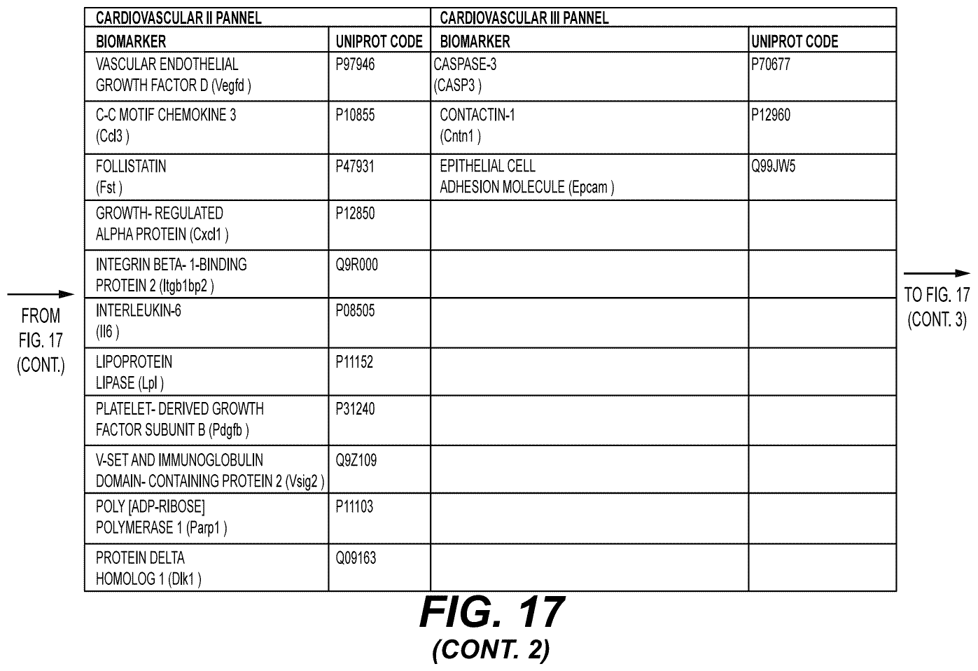

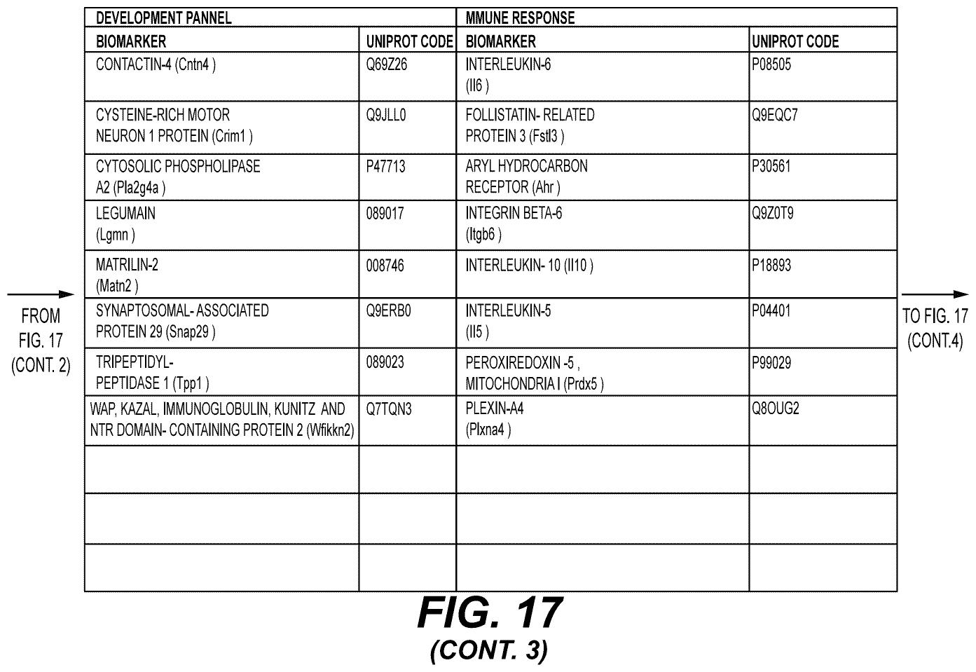

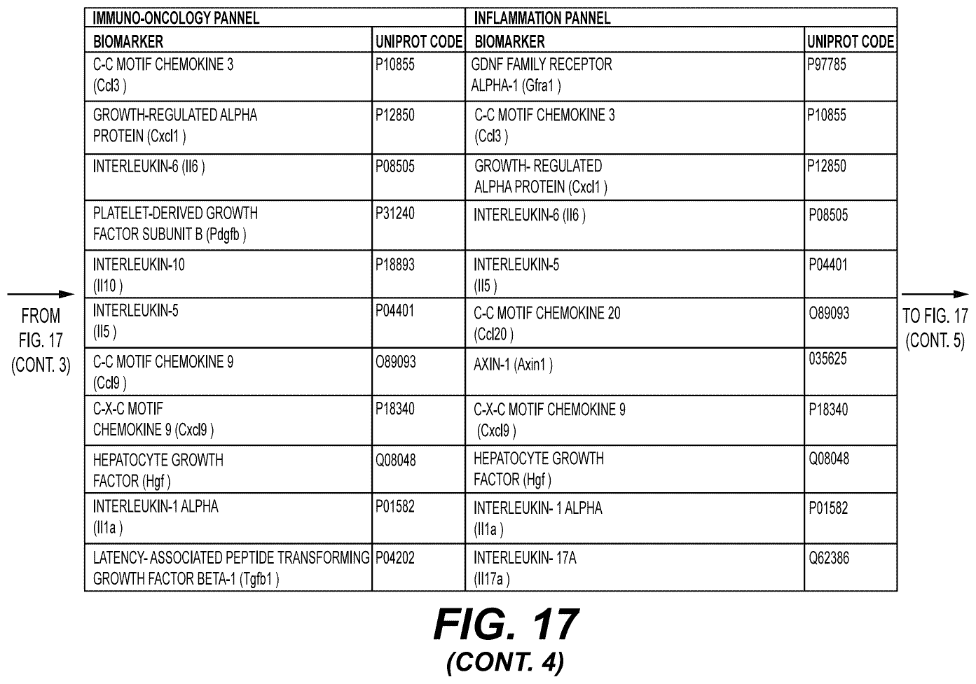

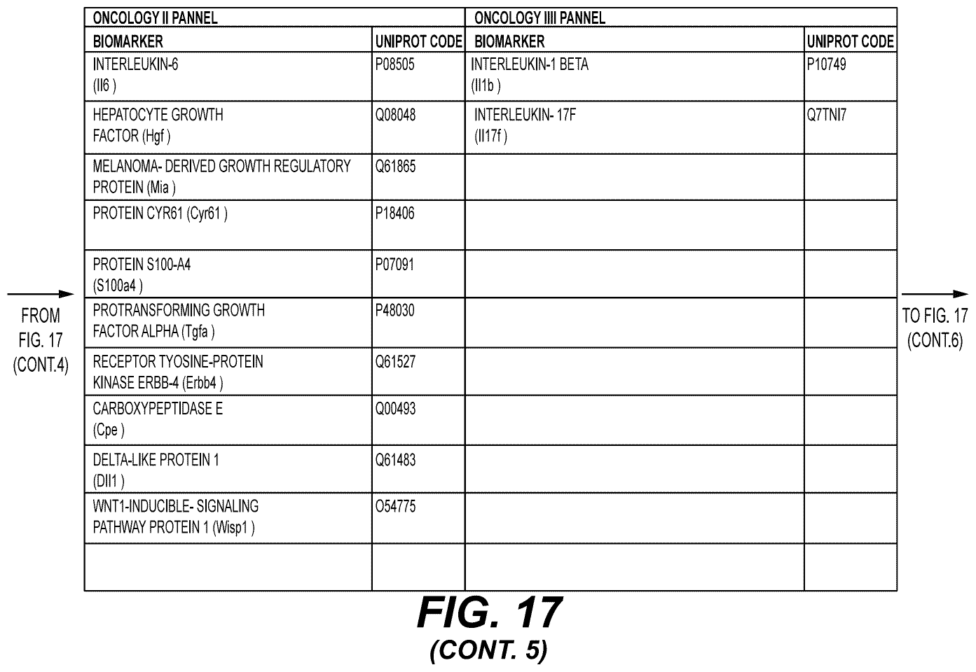

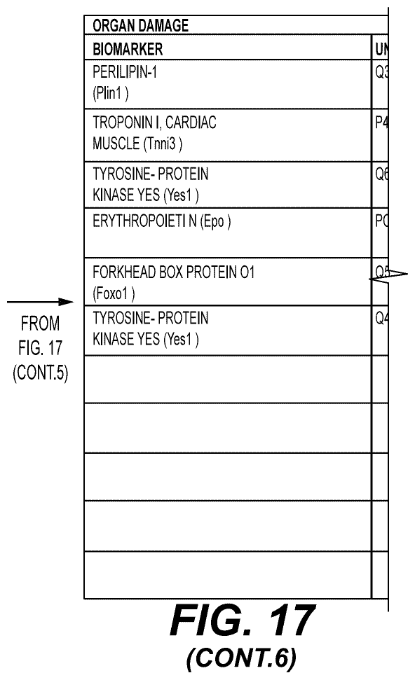

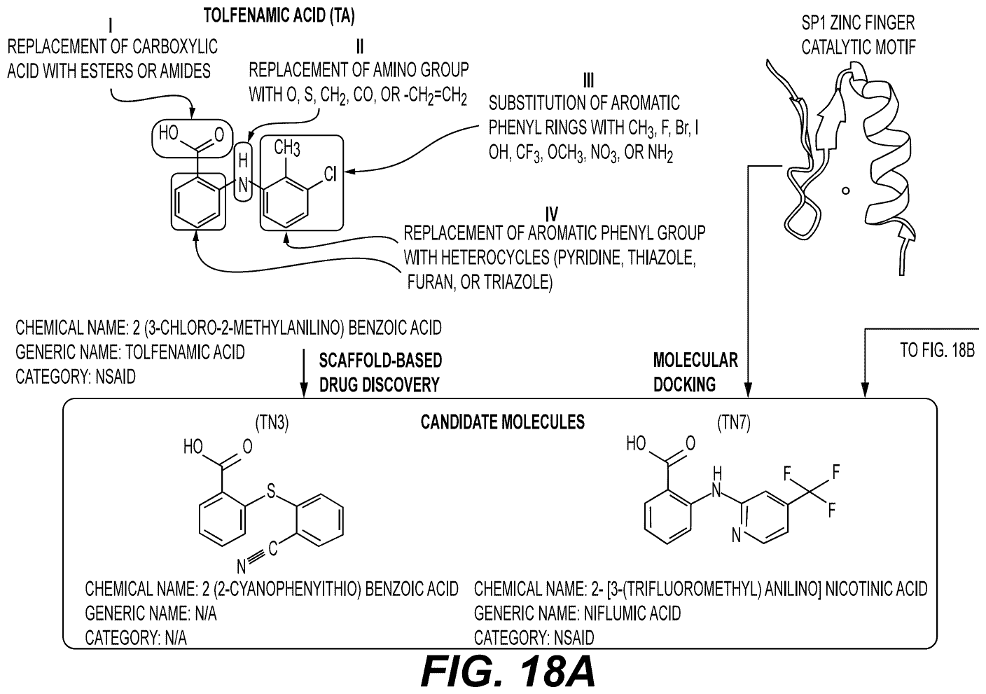

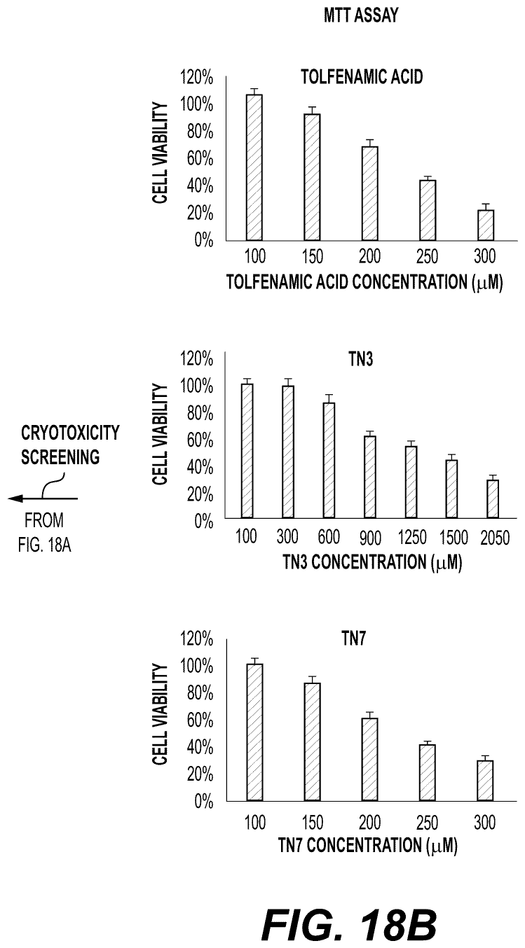

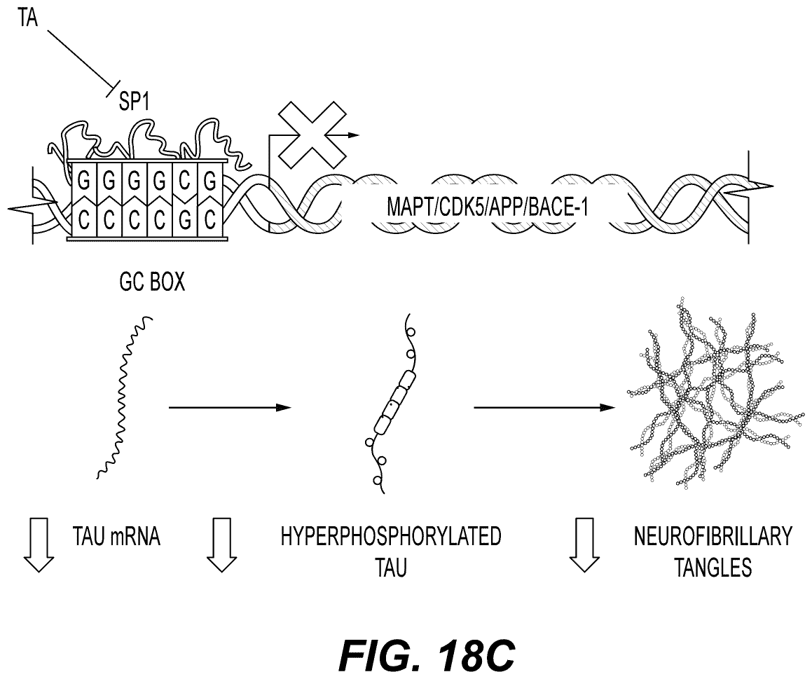

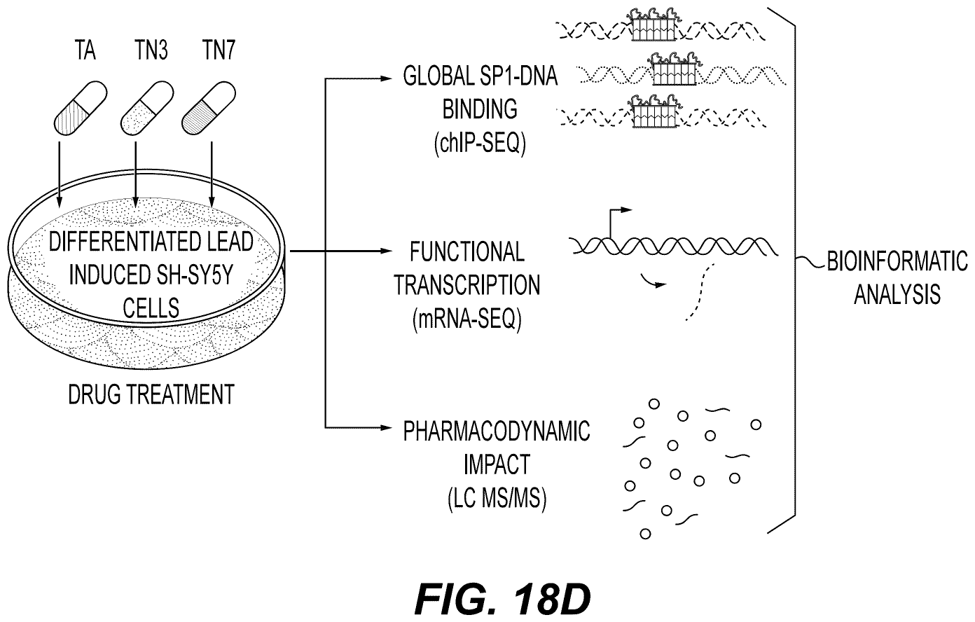

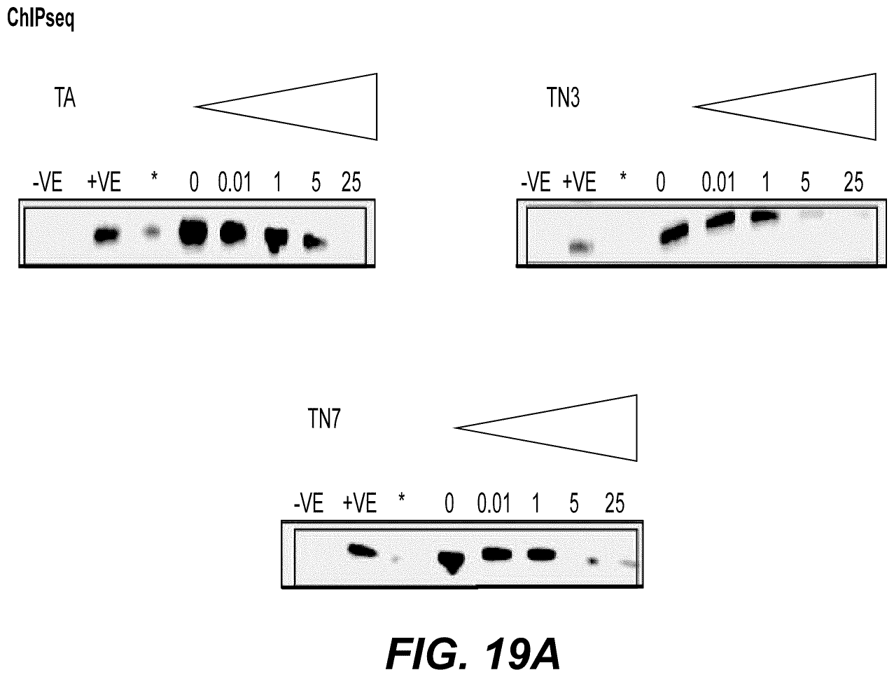

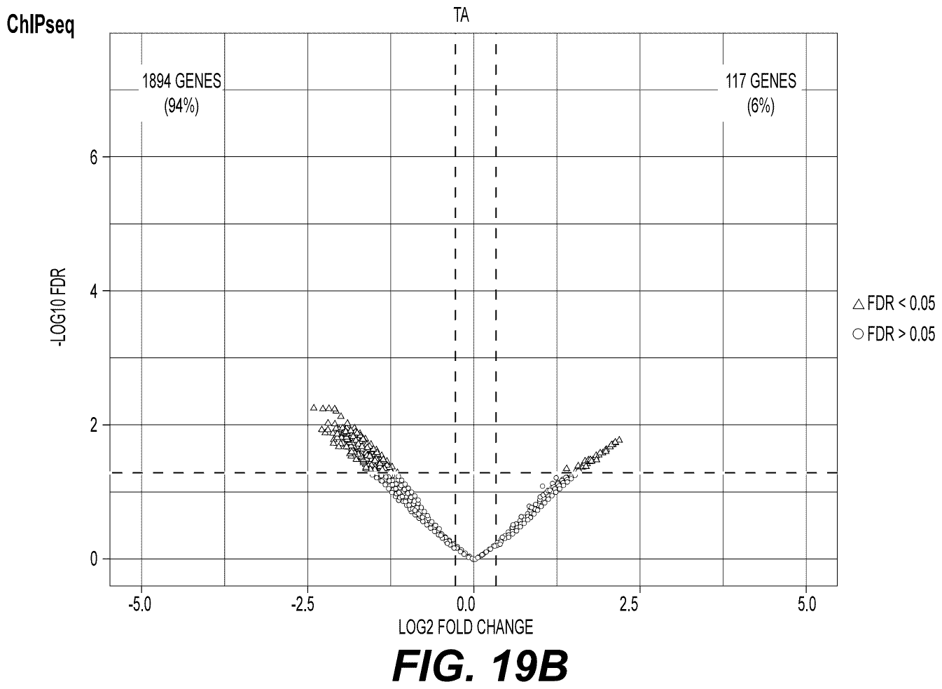

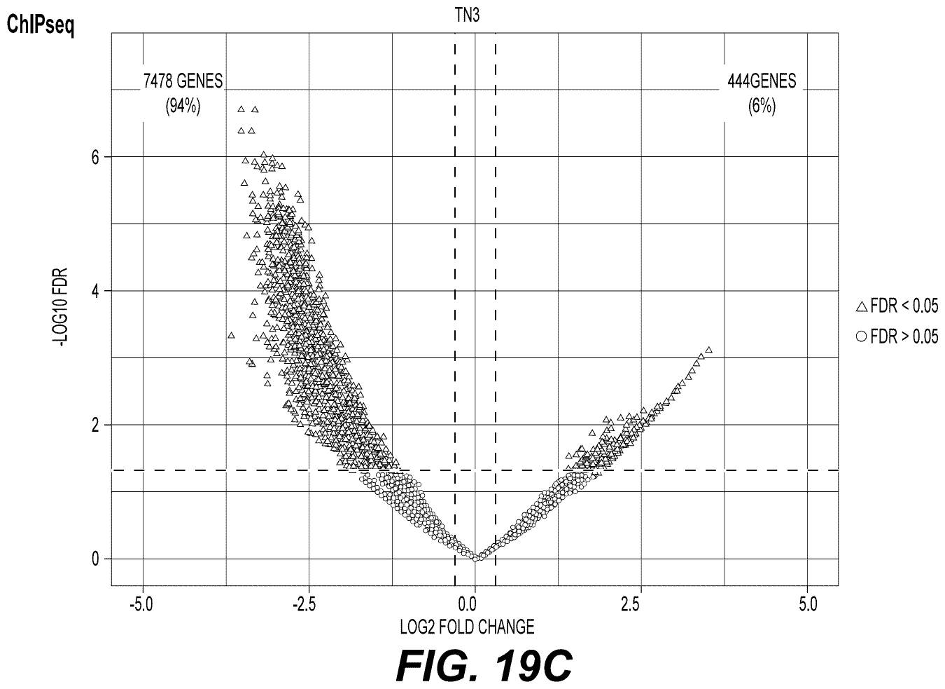

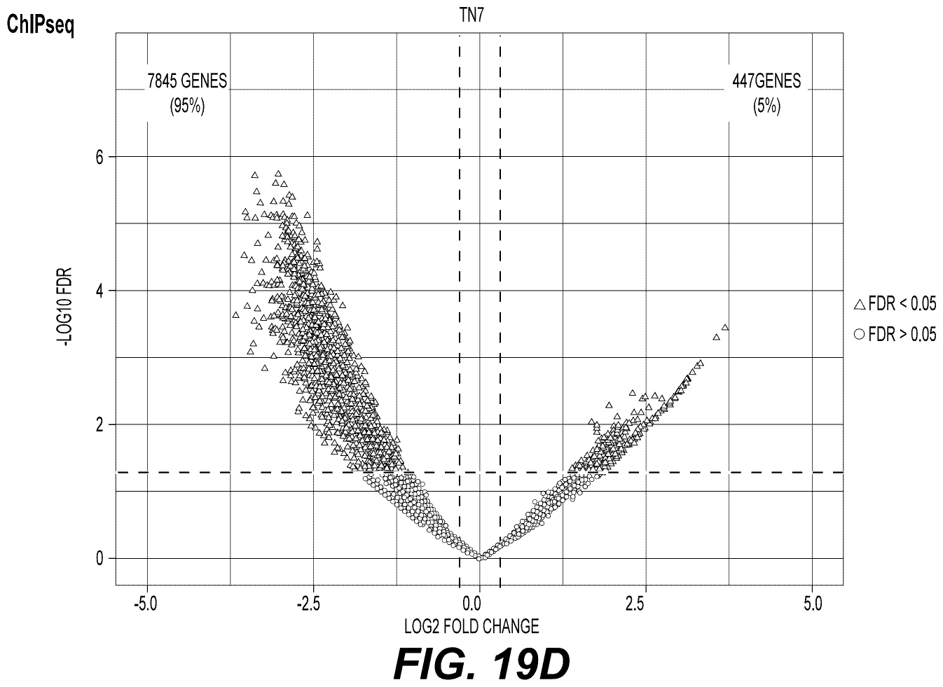

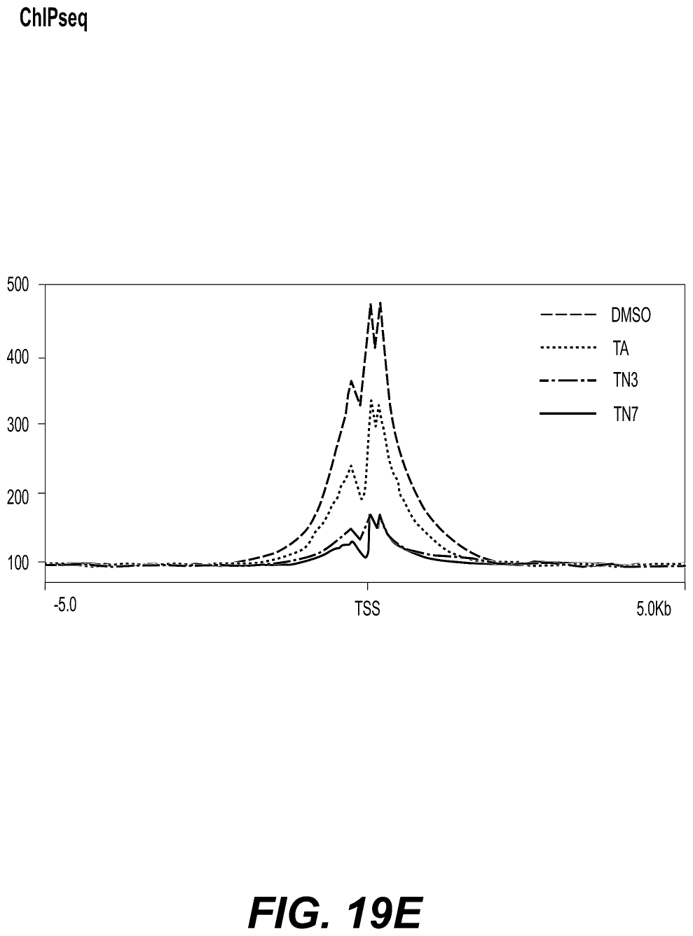

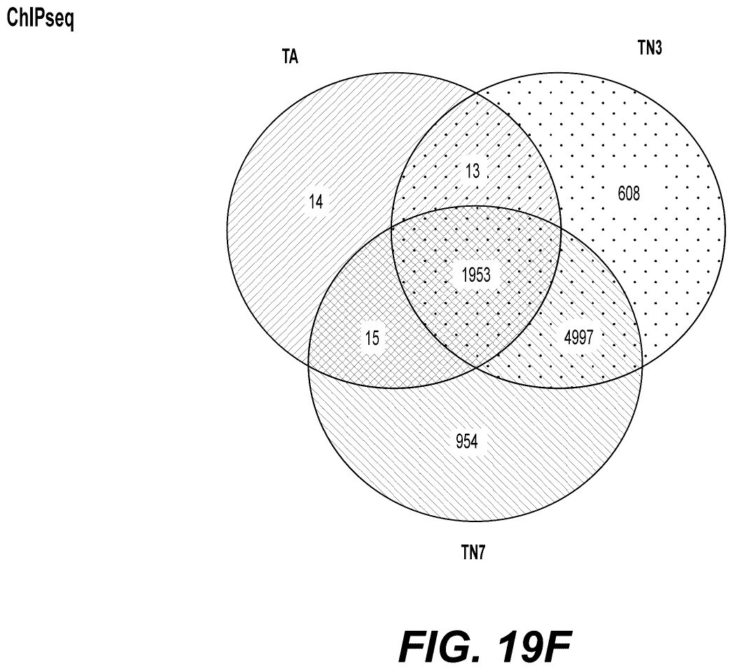

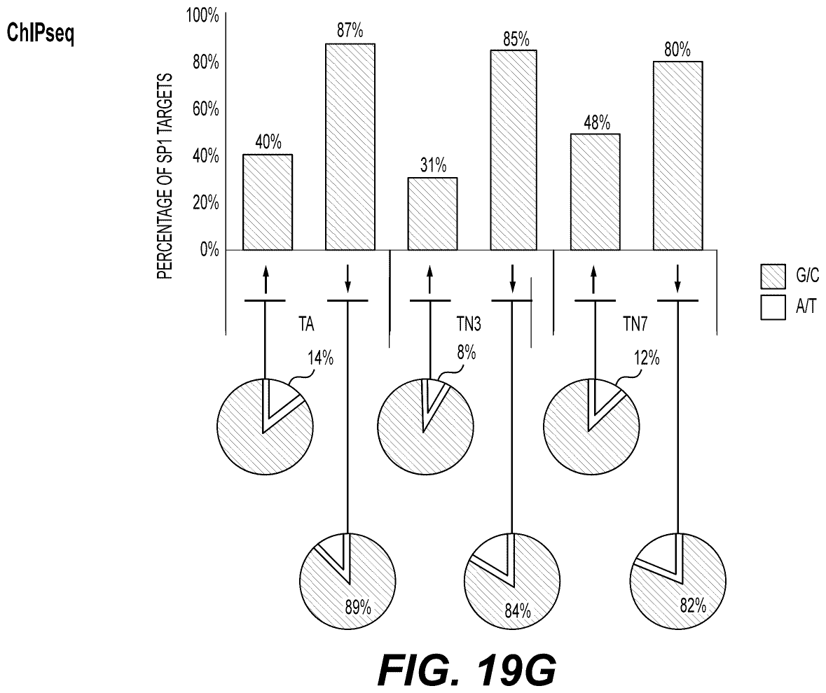

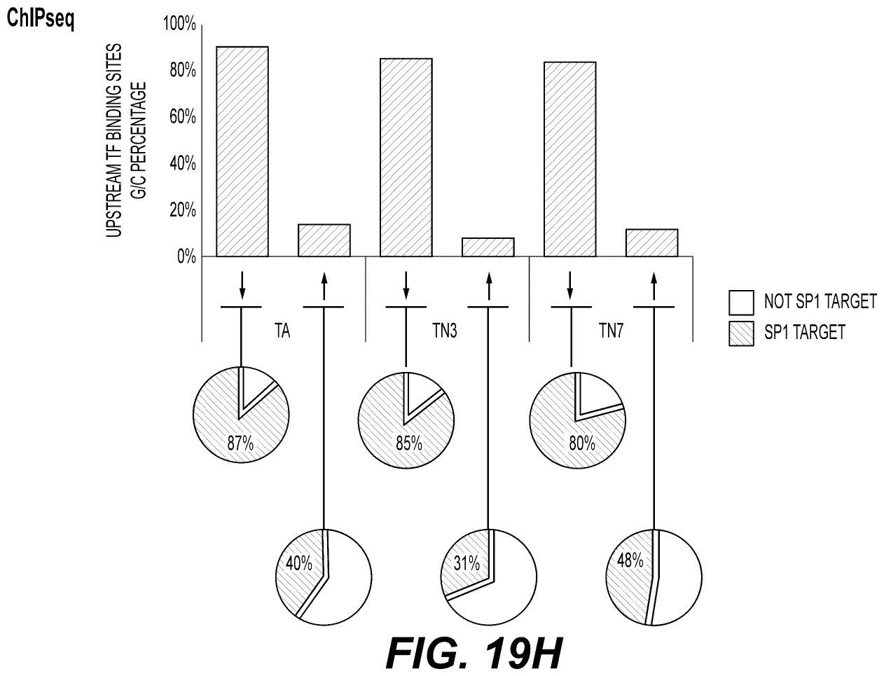

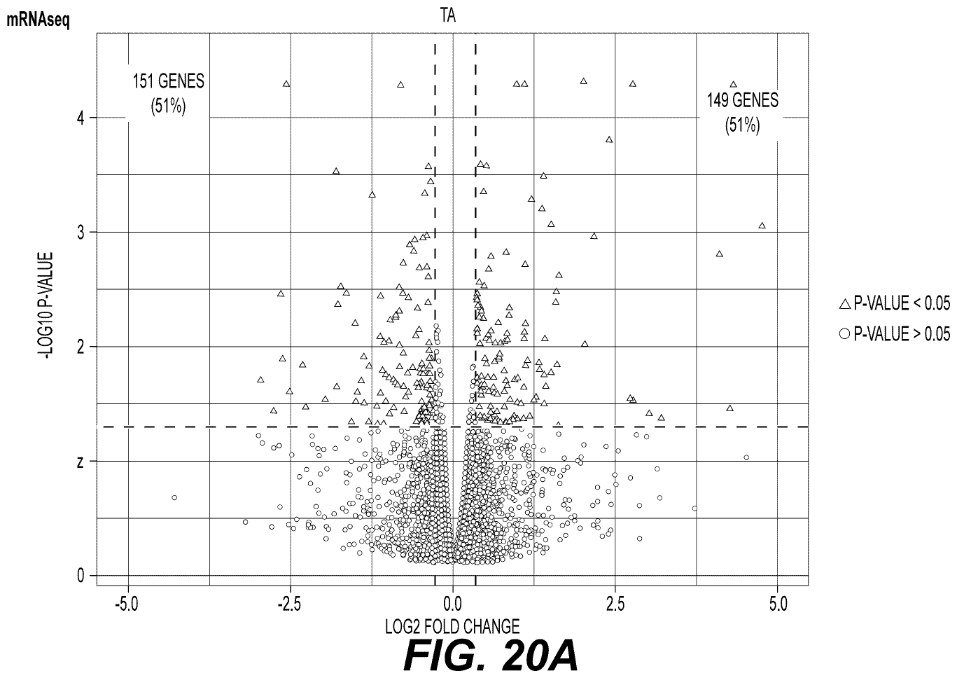

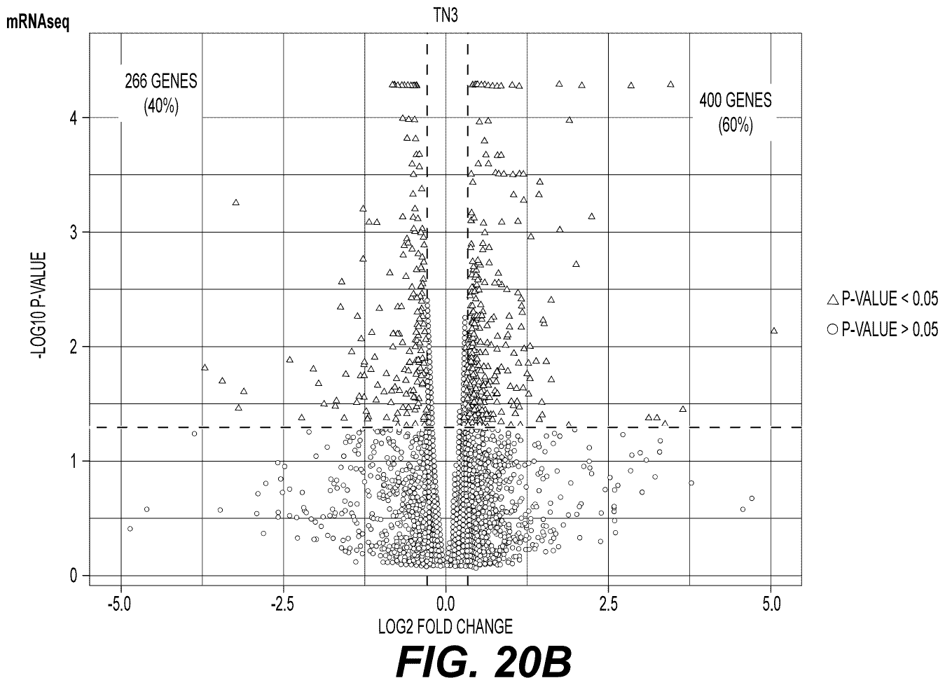

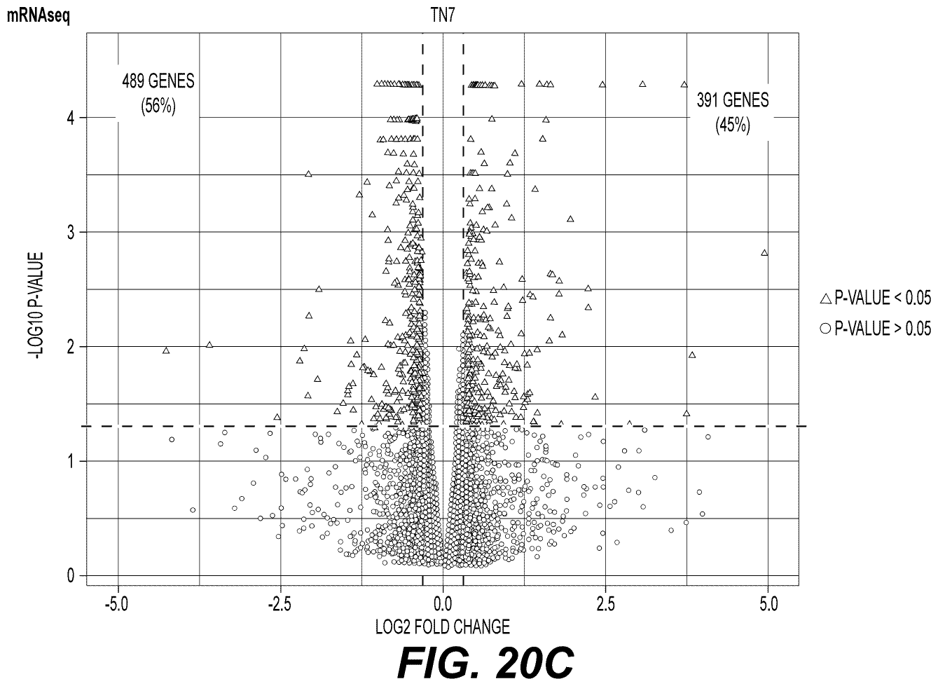

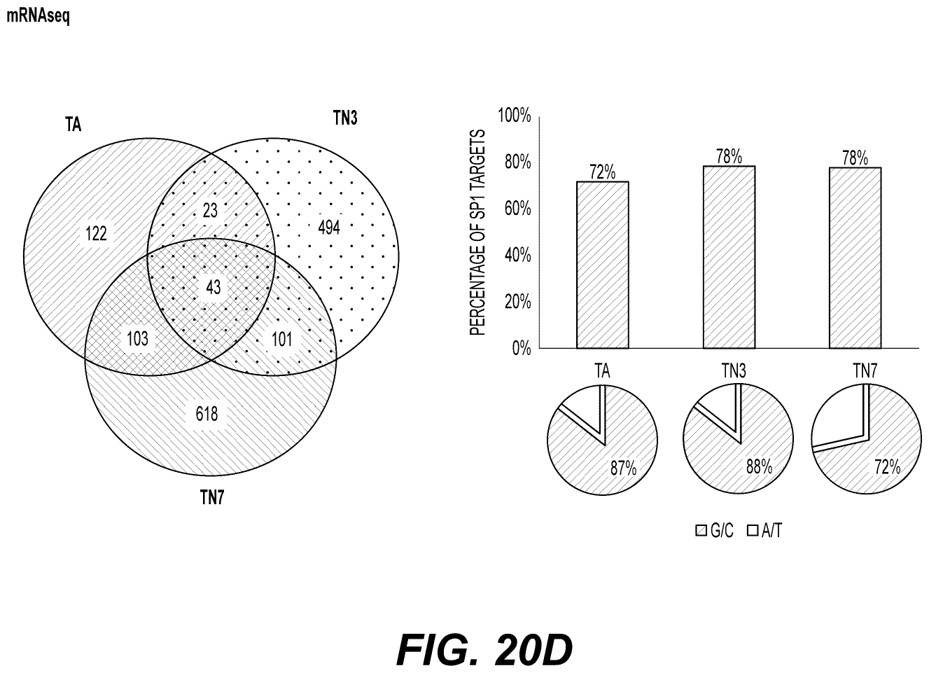

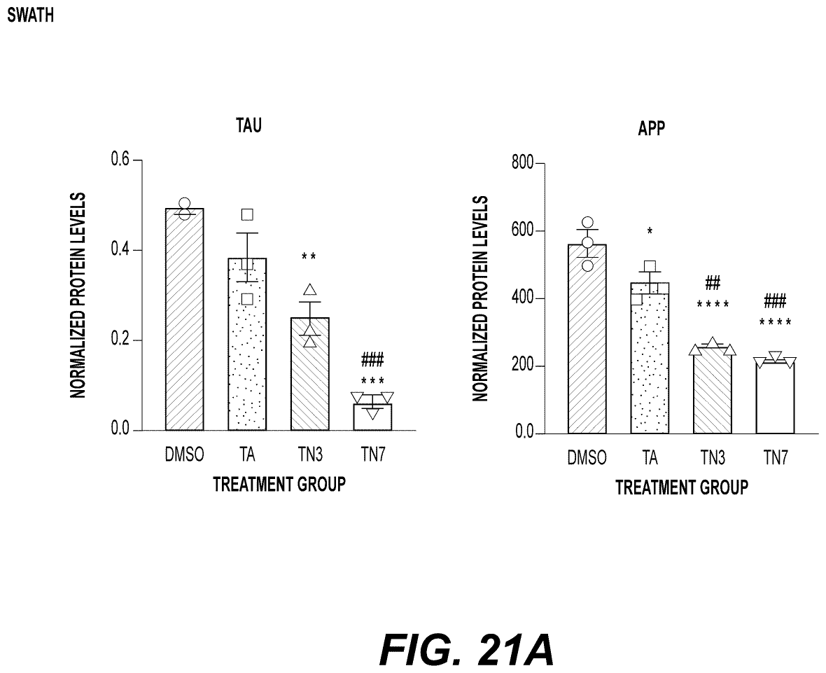

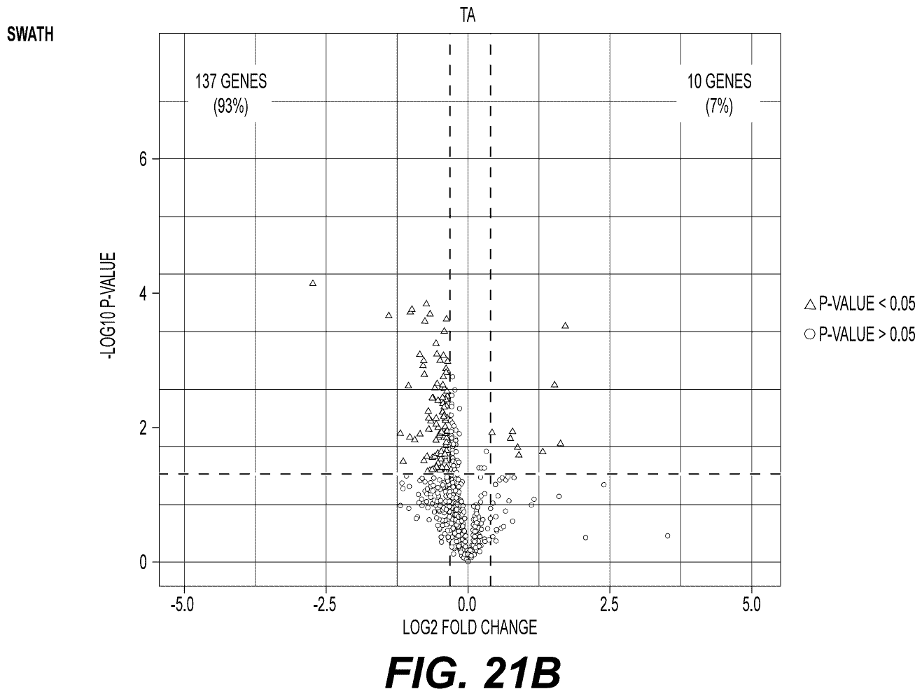

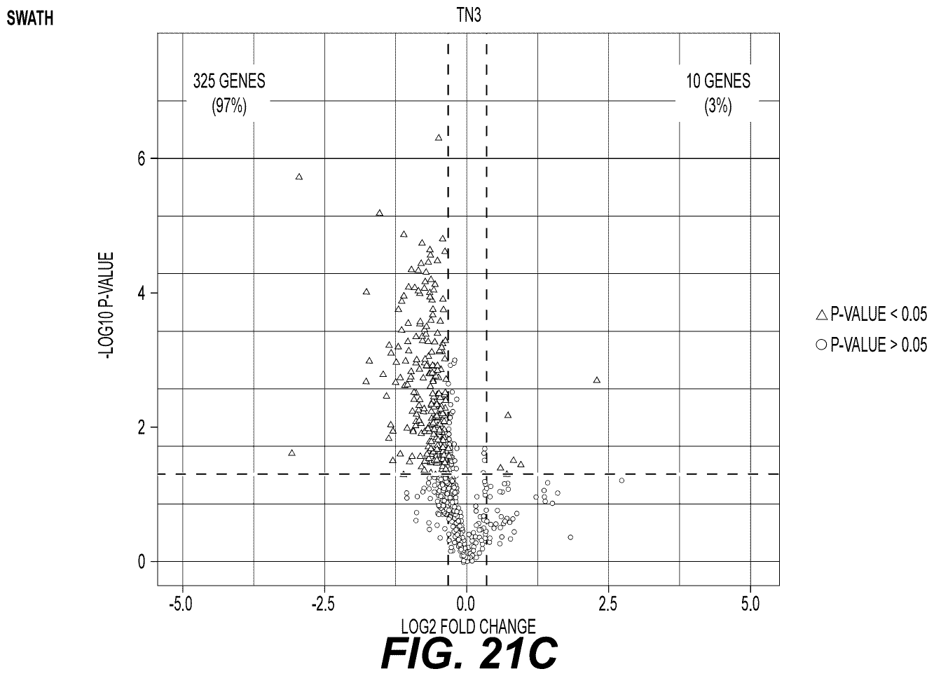

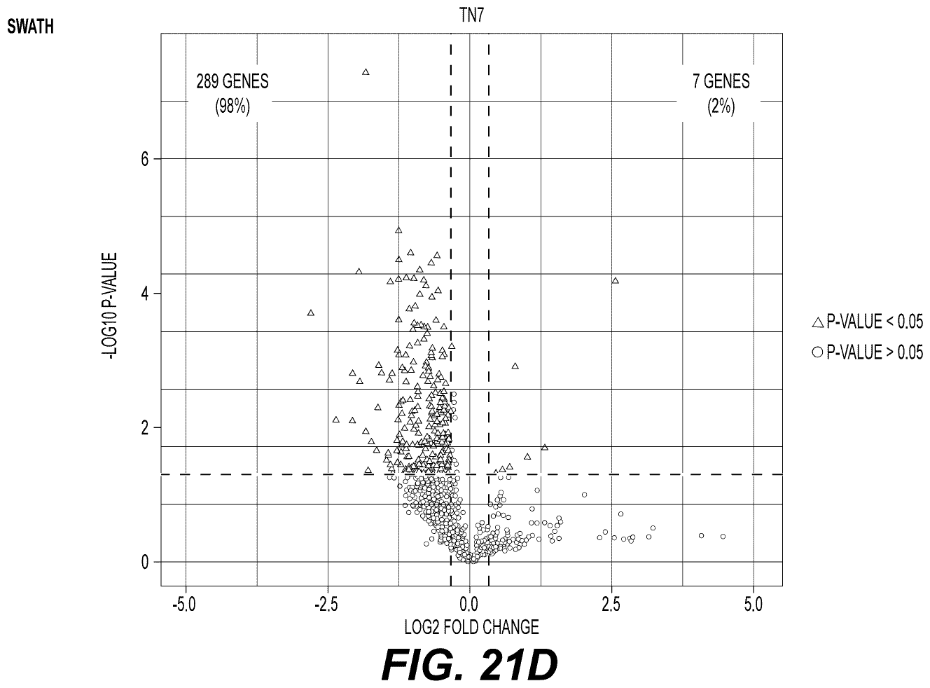

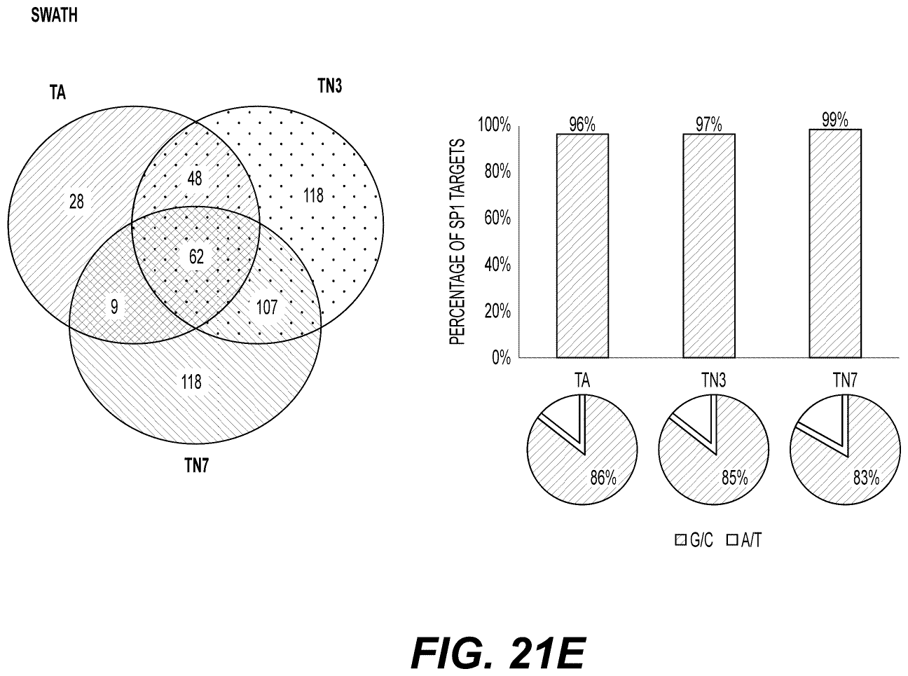

### DETAILED DESCRIPTION

SP1 is a zinc finger protein transcription factor that regulates the expression of the APP and Tau genes. As a zinc finger protein, SP1 uses zinc to stabilize its secondary structure, thus, disrupting the structure of SP1 would render its ability to bind to DNA and, therefore, lower AD and other neurodegeneration related biomarkers. Tolfenamic Acid (TA) or CLOTAM® Rapid is an NSAID that is currently used in Europe for the treatment of migraine headaches. TA is a unique drug among other NSAIDs due to its ability to cross the blood brain barrier (BBB) and interact directly with SP1.

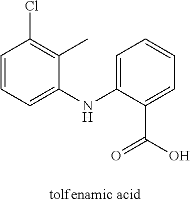 [SMILES: Cc1c(Cl)cccc1Nc1ccccc1C(=O)O]

According to the data, TA possibly chelates zinc from SP1 causing structural instability and loss of its DNA-binding ability. Using TA as a scaffold, analogs, that may be safer and more target-effective for the same AD-related biomarkers, were synthesized and investigated.

Aspects of the present disclosure are directed to methods of treating a neurodegenerative disease in a patient in need thereof. The methods include administering to the patient a therapeutically effective amount of a compound of Formula I

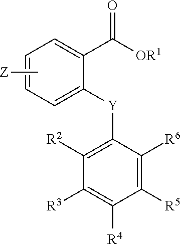

or a pharmaceutically acceptable salt thereof,
wherein Y is S, NH, O or CH₂;
R¹ is H or C₁₋₆ alky; and
R², R³, R⁴, R⁵, R⁶, and Z are each independently H, F, Cl, Br, NO₂, CF₃, CN, C₁₋₆ alky, or OCₙH₂ₙ₊₁,
wherein n is 1 to 6.

In some embodiments, Y is S; R¹, R³, R⁴, R⁵, R⁶ and Z are H; and R² is CN.

In some embodiments, Y is S; R¹, R² and Z are H; and one of R³, R⁴, R⁵, or R⁶ is CN.

In some embodiments, the compound of Formula 1 includes 2-(2-cyanophenylthio)benzoic acid, methyl 2-(2-cyanophenylthio)benzoate, ethyl 2-(2-cyanophenylthio)benzoate, 2-(2-chlorophenylthio)benzoic acid, 2-(2-nitrophenylthio)benzoic acid, 2-(2-methylphenylthio)benzoic acid, 2-(4-methylphenylthio)benzoic acid, 2-(4-fluorophenylthio)benzoic acid, 2-(4-chlorophenylthio)benzoic acid, 2-(4-nitrophenylthio)benzoic acid, 2-(3-methylphenylthio)benzoic acid, 2-(3-fluorophenylthio)benzoic acid, 2-(3-chlorophenylthio)benzoic acid, 2-(3-nitrophenylthio)benzoic acid, N-phenylanthranilic acid, N-(4-chlorophenyl)anthranilic acid, N-(4-hydroxyphenyl)anthranilic acid, or a pharmaceutically acceptable salt thereof. (see Table 1).

TABLE 1

| | |
|---|---|
| 2-(2-cyanophenylthio)benzoic acid | 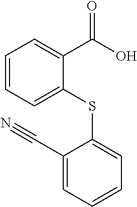 [SMILES: N#Cc1ccccc1Sc1ccccc1C(=O)O] |
| methyl 2-(2-cyanophenyl- thio)benzoate | 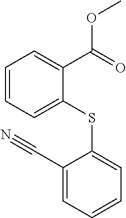 [SMILES: COC(=O)c1ccccc1Sc1ccccc1C#N] |
| ethyl 2-(2-cyanophenyl- thio)benzoate | 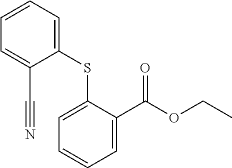 [SMILES: CCOC(=O)c1ccccc1Sc1ccccc1C#N] |
| 2-(2-chlorophenylthio)benzoic acid | 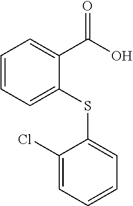 [SMILES: O=C(O)c1ccccc1Sc1ccccc1Cl] |
| 2-(2-nitrophenylthio)benzoic acid | 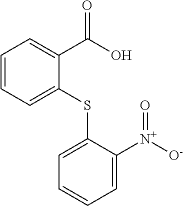 [SMILES: O=C(O)c1ccccc1Sc1ccccc1[N+](=O)[O-]] |
| 2-(2-methylphenylthio)benzoic acid | 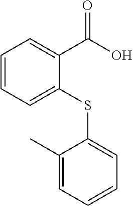 [SMILES: Cc1ccccc1Sc1ccccc1C(=O)O] |
| 2-(4-methylphenylthio)benzoic acid | 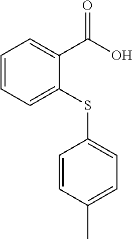 [SMILES: Cc1ccc(Sc2ccccc2C(=O)O)cc1] |
| 2-(4-fluorophenylthio)benzoic acid | 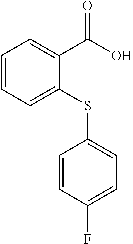 [SMILES: O=C(O)c1ccccc1Sc1ccc(F)cc1] |
| 2-(4-chlorophenylthio)benzoic acid | 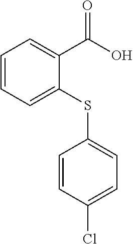 [SMILES: O=C(O)c1ccccc1Sc1ccc(Cl)cc1] |
| 2-(4-nitrophenylthio)benzoic acid | 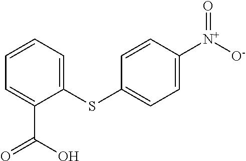 [SMILES: O=C(O)c1ccccc1Sc1ccc([N+](=O)[O-])cc1] |
| 2-(3-methylphenylthio)benzoic acid | 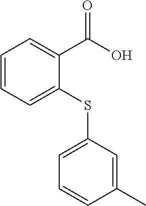 [SMILES: Cc1cccc(Sc2ccccc2C(=O)O)c1] |
| 2-(3-fluorophenylthio)benzoic acid | 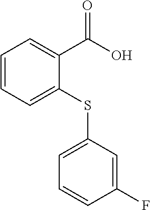 [SMILES: O=C(O)c1ccccc1Sc1cccc(F)c1] |
| 2-(3-chlorophenylthio)benzoic acid | 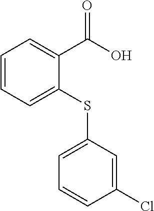 [SMILES: O=C(O)c1ccccc1Sc1cccc(Cl)c1] |
| 2-(3-nitrophenylthio)benzoic acid | 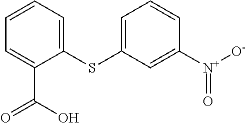 [SMILES: O=C(O)c1ccccc1Sc1cccc([N+](=O)[O-])c1] |
| N-phenylanthranilic acid | 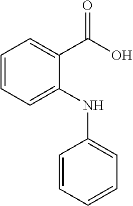 [SMILES: O=C(O)c1ccccc1Nc1ccccc1] |
| N-(4-chlorophenyl)anthranilic acid | 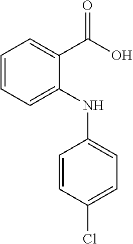 [SMILES: O=C(O)c1ccccc1Nc1ccc(Cl)cc1] |
| N-(4-hydroxyphenyl)anthranilic acid | 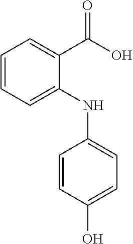 [SMILES: O=C(O)c1ccccc1Nc1ccc(O)cc1] |

In some embodiments, the neurodegenerative disease includes a tauopathy.

In some embodiments, the tauopathy includes one of Alzheimer's Disease (AD), chronic traumatic encephalopathy (CTE), traumatic brain injury (TBI), corticobasal degeneration, frontotemporal lobar degeneration (FTLD), behavioral variant frontotemporal dementia (bvFTLD), Pick disease, progressive supranuclear palsy (PSP), Down's Syndrome, Parkinson's disease, Primary Age-Related Tauopathy (PART), Globular Glial Tauopathy (GGT), Multiple System Atrophy (MSA), Argyrophilic grain disease (AGD), or Guam Parkinsonism-Dementia Complex.

In some embodiments, the tauopathy includes Alzheimer's Disease (AD).

In some embodiments, the tauopathy includes chronic traumatic encephalopathy (CTE).

In some embodiments, the tauopathy includes corticobasal degeneration.

In some embodiments, the tauopathy includes frontotemporal lobar degeneration (FTLD).

In some embodiments, the tauopathy includes behavioral variant frontotemporal dementia (bvFTLD).

In some embodiments, the tauopathy includes Pick disease.

In some embodiments, the tauopathy includes progressive supranuclear palsy (PSP).

In some embodiments, the treating includes reducing the rate of progression of the neurodegenerative disease.

In some embodiments, the treating includes reducing a symptom of the neurodegenerative disease.

Another aspect of the present disclosure is directed to pharmaceutical compositions including a compound of Formula I or a pharmaceutically acceptable salt thereof and a pharmaceutically acceptable carrier,

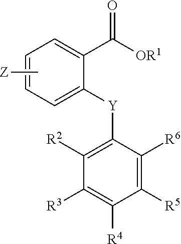

wherein Y is S, NH, O or CH₂;
R¹ is H or C₁₋₆ alky; and
R², R³, R⁴, R⁵, R⁶, and Z are each independently H, F, Cl, Br, NO₂, CF₃, CN, C₁₋₆ alky, or OCₙH₂ₙ₊₁,
wherein n is 1 to 6.

In some embodiments, Y is S; R¹, R³, R⁴, R⁵, R⁶ and Z are H; and R² is CN.

In some embodiments, Y is S; R¹, R² and Z are H; and one of R³, R⁴, R⁵, or R⁶ is CN.

In some embodiments, the pharmaceutical composition includes a solid dosage form.

In some embodiments, the pharmaceutical composition includes a liquid dosage form.

In some embodiments, the compound of Formula 1 includes 2-(2-cyanophenylthio)benzoic acid, methyl 2-(2-cyanophenylthio)benzoate, ethyl 2-(2-cyanophenylthio)benzoate, 2-(2-chlorophenylthio)benzoic acid, 2-(2-nitrophenylthio)benzoic acid, 2-(2-methylphenylthio)benzoic acid, 2-(4-methylphenylthio)benzoic acid, 2-(4-fluorophenylthio)benzoic acid, 2-(4-chlorophenylthio)benzoic acid, 2-(4-nitrophenylthio)benzoic acid, 2-(3-methylphenylthio)benzoic acid, 2-(3-fluorophenylthio)benzoic acid, 2-(3-chlorophenylthio)benzoic acid, 2-(3-nitrophenylthio)benzoic acid, N-phenylanthranilic acid, N-(4-chlorophenyl)anthranilic acid, N-(4-hydroxyphenyl)anthranilic acid, or a pharmaceutically acceptable salt thereof. (see Table 1).

Another aspect of the present disclosure is directed to methods of lowering tau or Amyloid Precursor Protein (APP) levels in a patient in need thereof by interrupting an upstream transcriptional pathway. The methods include administering to the patient a therapeutically effective amount of a compound of Formula I or a pharmaceutically acceptable salt thereof,

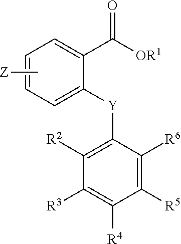

wherein Y is S, NH, O or CH₂;
R¹ is H or C₁₋₆ alky; and
R², R³, R⁴, R⁵, R⁶, and Z are each independently H, F, Cl, Br, NO₂, CF₃, CN, C₁₋₆ alky, or OCₙH₂ₙ₊₁,
wherein n is 1 to 6.

In some embodiments, Y is S; R¹, R³, R⁴, R⁵, R⁶ and Z are H; and R² is CN.

In some embodiments, Y is S; R¹, R² and Z are H; and one of R³, R⁴, R⁵, or R⁶ is CN.

In some embodiments, the therapeutically effective amount of the compound of Formula I or a pharmaceutically acceptable salt thereof includes a solid dosage form.

In some embodiments, the therapeutically effective amount of the compound of Formula I or a pharmaceutically acceptable salt thereof includes a liquid dosage form.

In some embodiments, the compound of Formula 1 includes 2-(2-cyanophenylthio)benzoic acid, methyl 2-(2-cyanophenylthio)benzoate, ethyl 2-(2-cyanophenylthio)benzoate, 2-(2-chlorophenylthio)benzoic acid, 2-(2-nitrophenylthio)benzoic acid, 2-(2-methylphenylthio)benzoic acid, 2-(4-methylphenylthio)benzoic acid, 2-(4-fluorophenylthio)benzoic acid, 2-(4-chlorophenylthio)benzoic acid, 2-(4-nitrophenylthio)benzoic acid, 2-(3-methylphenylthio)benzoic acid, 2-(3-fluorophenylthio)benzoic acid, 2-(3-chlorophenylthio)benzoic acid, 2-(3-nitrophenylthio)benzoic acid, N-phenylanthranilic acid, N-(4-chlorophenyl)anthranilic acid, N-(4-hydroxyphenyl)anthranilic acid, or a pharmaceutically acceptable salt thereof. (see Table 1).

Another aspect of the present disclosure is directed to kits that contain the disclosed formulations/compounds/compositions. For example, a disclosed kit can include a pharmaceutically acceptable carrier; a TA analog, and instructions for using the kit in a method for attenuating an immune response. The kit may also optionally include a means of administering the composition, for example, by injection.

### EXAMPLES

### Example 1—Production of TA Analogs

Using TA as a scaffold, analogs that may be safer and more target-effective for the same AD-related biomarkers were synthesized and investigated.

The synthesis of these compounds was accomplished using appropriately substituted benzoic acid and aniline derivatives, or other substituted aromatic or heteroaromatic compounds.

Synthetic Scheme

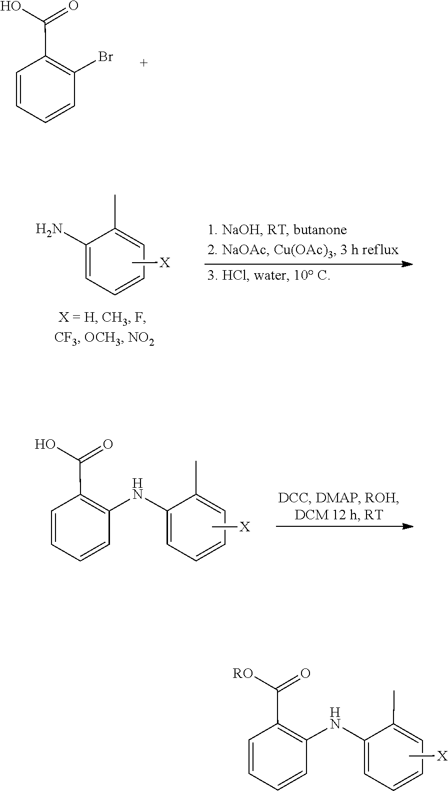

The foregoing scheme used the reaction of benzoic acid containing an appropriate leaving group at ortho position, such as —Br, with substituted aniline derivatives for the synthesis of compounds with diversified substituents. Esterification of TA derivatives with the corresponding alcohol in the presence of 2,2-dicyclohexylcarbodiimide (DCC) and catalytic amounts of 4-dimethylaminopyridine according to previously reported procedures generated ester derivatives. Similarly, ether and thiol derivatives were prepared by using phenol or thiophenol derivatives instead of aniline derivatives.

### Example 2—Testing TA Analogs

Differentiated SH-SY5Y cells were exposed with 25 μM lead (Pb acetate) to prime their neurodegenerative response. Then, the cells were treated with 0, 0.001 μM, 0.1 μM, 1 μM, 5 μM and 25 μM concentrations of Tolfenamic Acid (TA) and analogs. Cell viability assays were conducted using the MTA assay. Whole protein lysates were collected and Electrophoretic Mobility Shift Assays (EMSA) were conducted to determine the ability of compounds to bind SP1. Then Using Western blot analysis, the impact on the levels of protein biomarkers associated with neurodegeneration: SP1, APP, Tau, Phopsho Tau, and GSK3beta were examined.

Results: Two analogs showed better a safety profile than TA and decreased SP1-DNA binding at 48 and 72 hours. SP1 levels also decreased. Consequently, the gene products regulated by SP1 exhibited decreases in their levels (APP, Tau, pTau, GSK3beta).

Conclusion: Targeting an upstream transcription factor is believed to be essential to modulate the expression of neurodegeneration related genes and maybe a promising way to halt disease progression. These analogs target SP1 and appear to be therapeutics that are safer and more effective than TA.

### Example 3—Treatment of an AD Patient

A therapeutically effective amount of the TA analog 2-(2-cyanophenylthio) benzoic acid is administered to an AD patient. The patient's symptoms are reduced as compared to a non-treated patient.

### Example 4—Treatment of a Chronic Traumatic Encephalopathy (CTE) Patient

A therapeutically effective amount of the TA analog 2-(2-cyanophenylthio) benzoic acid is administered to a CTE patient. The patient's symptoms are reduced as compared to a non-treated patient.

### Example 5—Treatment of a Corticobasal Degeneration Patient

A therapeutically effective amount of the TA analog 2-(2-cyanophenylthio) benzoic acid is administered to a corticobasal degeneration patient. The patient's symptoms are reduced as compared to a non-treated patient.

### Example 6—Treatment of a Frontotemporal Lobar Degeneration (FTLD) Patient

A therapeutically effective amount of the TA analog 2-(2-cyanophenylthio) benzoic acid is administered to an FTLD patient. The patient's symptoms are reduced as compared to a non-treated patient.

### Example 7—Treatment of a Behavioral Variant Frontotemporal Dementia (bvFTLD) Patient

A therapeutically effective amount of the TA analog 2-(2-cyanophenylthio) benzoic acid is administered to a bvFTLD patient. The patient's symptoms are reduced as compared to a non-treated patient.

### Example 8—Treatment of a Pick Disease Patient

A therapeutically effective amount of the TA analog 2-(2-cyanophenylthio) benzoic acid is administered to a Pick disease patient. The patient's symptoms are reduced as compared to a non-treated patient.

### Example 9—Treatment of a Progressive Supranuclear Palsy (PSP) Patient

A therapeutically effective amount of the TA analog 2-(2-cyanophenylthio) benzoic acid is administered to a PSP patient. The patient's symptoms are reduced as compared to a non-treated patient.

### Example 10—Treatment of a Traumatic Brain Injury (TBI) Patient

A therapeutically effective amount of the TA analog 2-(2-cyanophenylthio) benzoic acid is administered to a TBI patient. The patient's symptoms are reduced as compared to a non-treated patient.

### Example 11—Treatment of a Down's Syndrome Patient

A therapeutically effective amount of the TA analog 2-(2-cyanophenylthio) benzoic acid is administered to a Down's Syndrome patient. The patient's symptoms are reduced as compared to a non-treated patient.

“A” and “an” are used herein to refer to one or to more than one (i.e., to at least one) of the grammatical object of the article. By way of example, “an element” means one element or more than one element.

“Comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

“In vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

“Or” as used herein should be understood to mean “and/or”, unless the context clearly indicates otherwise.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

The term “treatment” may refer to the application of one or more specific procedures used for the amelioration of a disease. In certain embodiments, the specific procedure is the administration of one or more pharmaceutical agents. “Treatment” of an individual (e.g. a mammal, such as a human) or a cell is any type of intervention used in an attempt to alter the natural course of the individual or cell. Treatment includes, but is not limited to, administration of a therapeutic agent or a pharmaceutical composition, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition being treated. Also included are “prophylactic” treatments, which can be directed to reducing the rate of progression of the disease or condition being treated, delaying the onset of that disease or condition, or reducing the severity of its onset.

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

“Reducing,” “suppressing” and “inhibiting” have their commonly understood meaning of lessening or decreasing.

As used herein, “pharmaceutically acceptable salts” refers to an ionizable therapeutic agent that has been combined with a counter-ion to form a neutral complex. Lists of suitable salts are found, for example, in Remington's Pharmaceutical Sciences, 17th ed., Mack Publishing Company, Easton, Pa., 1985, p. 1418, Journal of Pharmaceutical Science, 66, 2 (1977), and “Handbook of Pharmaceutical Salts: Properties, Selection, and Use” (P. Henrich Stahl & Camille G. Wermuth (Eds.), VHCA & Wiley-VCH, 2002).

The terms “pharmaceutical,” “pharmaceutically acceptable” and “therapeutically acceptable” may refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. These substances do not interfere with the effectiveness or the biological activity of the active ingredients.

“Pharmaceutically acceptable carrier” means a carrier that is useful for the preparation of a pharmaceutical composition that is: generally compatible with the other ingredients of the composition, not deleterious to the recipient, and neither biologically nor otherwise undesirable. “A pharmaceutically acceptable carrier” includes both one and more than one carrier. Embodiments include carriers for topical, ocular, parenteral, intravenous, intraperitoneal intramuscular, sublingual, nasal, and oral administration. “Pharmaceutically acceptable carrier” also includes agents for preparation of aqueous dispersions and sterile powders for injection or dispersions.

Pharmaceutically acceptable carriers, adjuvants and vehicles that may be used in the pharmaceutical compositions of the present disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present disclosure include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product. Also, see, for example, Ansel's Pharmaceutical Dosage Forms and Drug Delivery Systems, Wolters Kluwer Health (11th ed. 2018).

In some embodiments, any one of the compounds and therapeutic agents disclosed herein are administered orally. Compositions of the present disclosure suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats, and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

The pharmaceutical compositions of the present disclosure may be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present disclosure is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

Examples of useful dermatological compositions which can be used to deliver the compounds to the skin are known in the art; see for example, Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

The compounds and therapeutic agents of the present disclosure may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present disclosure provides an implantable drug release device impregnated with or containing a compound or a therapeutic agent, or a composition comprising a compound of the present disclosure or a therapeutic agent, such that said compound or therapeutic agent is released from said device and is therapeutically active.

Dosages and Regimens

In the pharmaceutical compositions of the present disclosure, 2-(2-cyanophenylthio)benzoic acid or an analog or derivative or a pharmaceutically acceptable salt thereof (and/or one or more of the other listed TA analogs or pharmaceutically acceptable salts thereof) is present in an effective amount (e.g., a therapeutically effective amount).

Effective doses may vary, depending on the diseases treated, the severity of the disease, the route of administration, the sex, age and general health condition of the subject, excipient usage, and the possibility of co-usage with other therapeutic treatments such as use of other agents and the judgment of the treating physician.

In some embodiments, an effective amount of a 2-(2-cyanophenylthio)benzoic acid or an analog or derivative or a pharmaceutically acceptable salt thereof (and/or one or more of the other listed TA analogs or pharmaceutically acceptable salts thereof) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of 2-(2-cyanophenylthio)benzoic acid or an analog or derivative or a pharmaceutically acceptable salt thereof (and/or one or more of the other listed TA analogs or pharmaceutically acceptable salts thereof) is about 0.1 mg/kg, about mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Kits

In some embodiments, provided herein are packaged dosage forms, comprising a container holding a therapeutically effective amount of 2-(2-cyanophenylthio)benzoic acid or an analog or derivative or a pharmaceutically acceptable salt thereof (and/or one or more of the other listed TA analogs or pharmaceutically acceptable salts thereof), and instructions for using the dosage form in accordance with one or more of the methods provided herein.

The present dosage forms and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, the described dosage forms can also be provided in kit form combined with other components necessary for administration of the material to the patient. For example, disclosed kits, such as for use in the treatments described herein, can further comprise, for example, administration materials.

The kits may be designed in various forms based on the specific deficiencies they are designed to treat.

The dosage forms provided herein may be prepared and placed in a container for storage at ambient or elevated temperature. This is beneficial because transportation of commercially viable dosage forms may benefit from stability at temperatures greater than those requiring refrigeration or sub-freezing environments during transportation and storage at the site of use.

When the dosage forms provided herein are stored in a polyolefin plastic container as compared to, for example, a polyvinyl chloride plastic container, discoloration of the dosage form may be reduced. Without wishing to be bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce adherence or adsorption of the active ingredient to the surface of the container, which could effectively dilute the concentration of active ingredient in the contained solution. Some containers also include the capacity to reduce exposure of the container's contents to infrared light, or a second component with such a capacity. Some containers further include the capacity to reduce the exposure of the container's contents to heat or humidity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a glass container. The container may further be disposed within a second container, for example, a paper container, cardboard container, paperboard container, metallic film container, or foil container, or a combination thereof, to further reduce exposure of the container's contents to UV, visible, or infrared light. Articles of manufacture benefiting from reduced discoloration, decomposition, or both during storage, include dosage forms that include 2-(2-cyanophenylthio)benzoic acid or an analog or derivative or a pharmaceutically acceptable salt thereof (and/or one or more of the other listed TA analogs or pharmaceutically acceptable salts thereof). The dosage forms provided herein may need storage lasting up to, or longer than, three months; in some cases up to, or longer than one year. The containers may be in any form suitable to contain the contents—for example, a bag, a bottle, or a box.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

The terms “effective,” “effective amount” or “therapeutically effective amount” refer to an amount, i.e., a dosage, of therapeutic agent administered to a subject (e.g., a mammalian subject, i.e., a human subject), either as a single dose or as part of a series of doses, which is effective to produce a desired therapeutic effect (e.g., effective for influencing, reducing or inhibiting the activity of or preventing activation of a kinase, or effective at bringing about a desired in vivo effect in an animal, preferably, a human, such as reduction in intraocular pressure).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method of treating a neurodegenerative disease in a patient in need thereof, comprising administering to the patient a therapeutically effective amount of a compound of Formula I 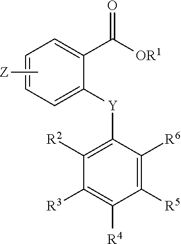
or a pharmaceutically acceptable salt thereof,
wherein Y is S, NH, O or CH₂;
R¹ is H or C₁₋₆ alky; and
R², R³, R⁴, R⁵, R⁶, and Z are each independently H, F, Cl, Br, NO₂, CF₃, CN, C₁₋₆ alky, or OCₙH₂ₙ₊₁,
wherein n is 1 to 6.

2. The method of claim 1, wherein Y is S; R¹, R³, R⁴, R⁵, R⁶ and Z are H; and R² is CN.

3. The method of claim 1, wherein Y is S; R¹, R² and Z are H; and one of R³, R⁴, R⁵, or R⁶ is CN.

4. The method of claim 1, wherein the compound of Formula I comprises 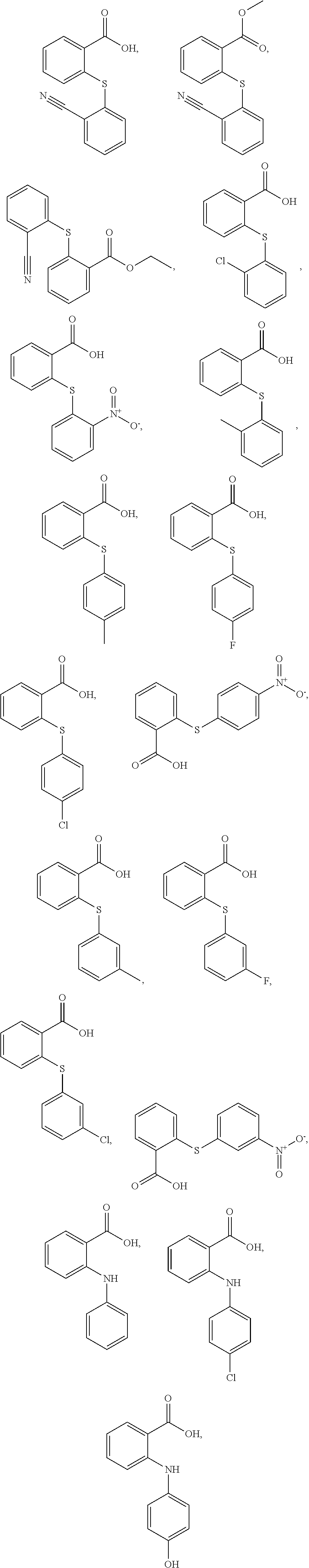
or a pharmaceutically acceptable salt thereof.

5. The method of claim 1, wherein said neurodegenerative disease comprises a tauopathy.

6. The method of claim 5, wherein said tauopathy comprises one of Alzheimer's Disease (AD), chronic traumatic encephalopathy (CTE), traumatic brain injury (TBI), corticobasal degeneration, frontotemporal lobar degeneration (FTLD), behavioral variant frontotemporal dementia (bvFTLD), Pick disease, progressive supranuclear palsy (PSP), Down's Syndrome, Parkinson's disease, Primary Age-Related Tauopathy (PART), Globular Glial Tauopathy (GGT), Multiple System Atrophy (MSA), Argyrophilic grain disease (AGD), or Guam Parkinsonism-Dementia Complex.

7. The method of claim 6, wherein said tauopathy comprises Alzheimer's Disease (AD).

8. The method of claim 6, wherein said tauopathy comprises chronic traumatic encephalopathy (CTE).

9. The method of claim 6, wherein said tauopathy comprises corticobasal degeneration.

10. The method of claim 6, wherein said tauopathy comprises frontotemporal lobar degeneration (FTLD).

11. The method of claim 1, wherein said treating comprises reducing the rate of progression of the neurodegenerative disease.

12. The method of claim 1, wherein said treating comprises reducing a symptom of the neurodegenerative disease.

13. A pharmaceutical composition comprising a of a compound of Formula I 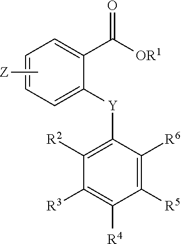
or and a pharmaceutically acceptable carrier,
wherein Y is S, NH, O or CH₂;
R¹ is H or C₁₋₆ alky; and
R², R³, R⁴, R⁵, R⁶, and Z are each independently H, F, Cl, Br, NO₂, CF₃, CN, C₁₋₆ alky, or OCₙH₂ₙ₊₁,
wherein n is 1 to 6.

14. The pharmaceutical composition of claim 13, wherein Y is S; R¹, R³, R⁴, R⁵, R⁶ and Z are H; and R² is CN.

15. The pharmaceutical composition of claim 13, wherein Y is S; R¹, R² and Z are H; and one of R³, R⁴, R⁵, or R⁶ is CN.

16. The pharmaceutical composition of claim 13, wherein the compound of Formula I comprises 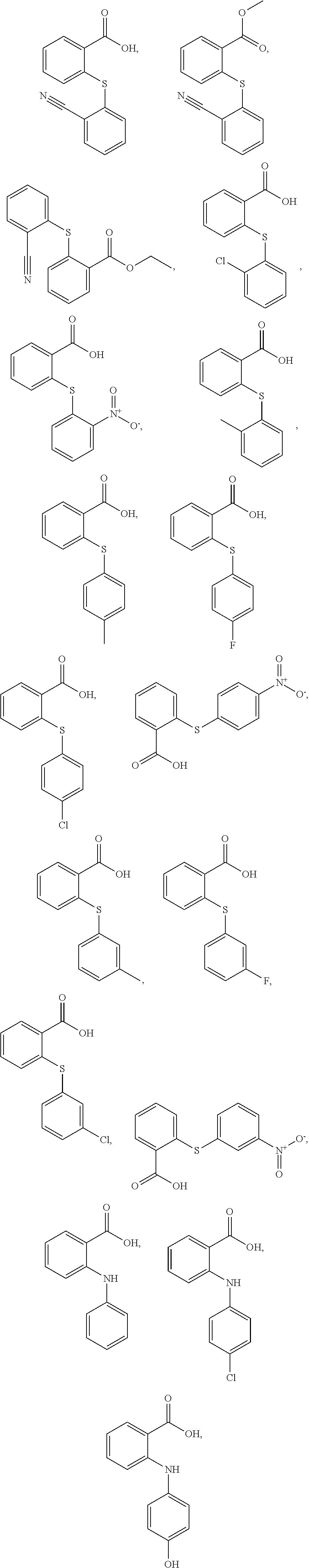
or a pharmaceutically acceptable salt thereof.

17. A method of lowering tau or Amyloid Precursor Protein (APP) levels in a patient in need thereof by interrupting an upstream transcriptional pathway, comprising administering to the patient a therapeutically effective amount of a compound of Formula I 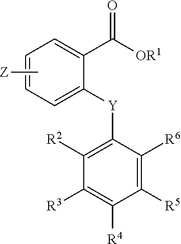
or a pharmaceutically acceptable salt thereof,
wherein Y is S, NH, O or CH₂;
R¹ is H or C₁₋₆ alky; and
R², R³, R⁴, R⁵, R⁶, and Z are each independently H, F, Cl, Br, NO₂, CF₃, CN, C₁₋₆ alky, or OCₙH₂ₙ₊₁,
wherein n is 1 to 6.

18. The method of claim 17, wherein Y is S; R¹, R³, R⁴, R⁵, R⁶ and Z are H; and R² is CN.

19. The method of claim 17, wherein Y is S; R¹, R² and Z are H; and one of R³, R⁴, R⁵, or R⁶ is CN.

20. The method of claim 17, wherein the compound of Formula I comprises 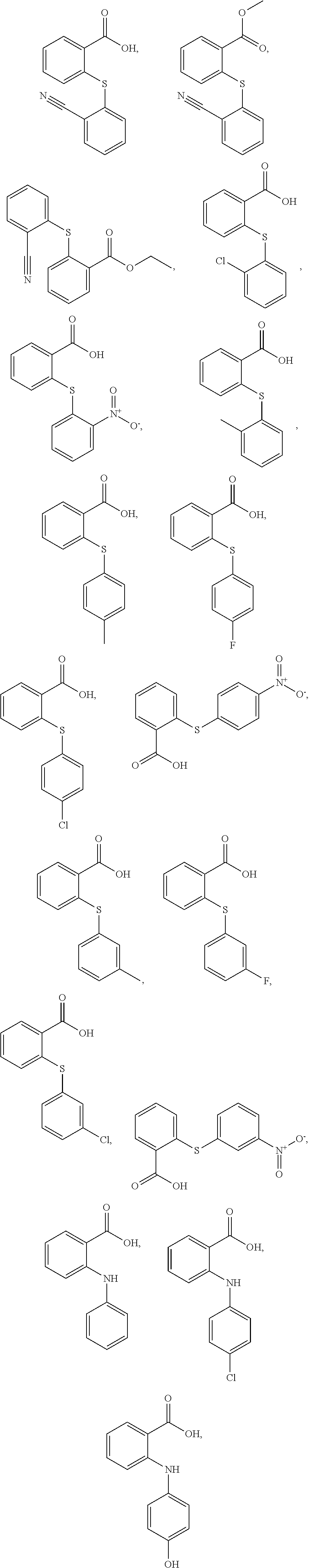
or a pharmaceutically acceptable salt thereof.
